(19) United States
(12) Patent Application Publication
(10) Pub. No.: US 2024/0206440 A1
(43) Pub. Date: Jun. 27, 2024

(54) ANIMAL MODELS AND THERAPEUTIC MOLECULES

(71) Applicant: PETMEDIX LTD., Cambridge (GB)

(72) Inventors: Albert Vilella BERTRAN, Cambridge (GB); Daniel John BOLLAND, Cambridge (GB)

(21) Appl. No.: 18/579,594

(22) PCT Filed: May 24, 2022
(86) PCT No.: PCT/EP2022/063997

§ 371 Date: Jan. 16, 2024

(30) Foreign Application Priority Data
May 24, 2021 (GB) 2107372.1

(51) Int. Cl. A01K 67/0275; A61K 39/00; C07K 16/18

(52) U.S. Cl. CPC A01K 67/0275; C07K 16/18; A01K 2217/072; A01K 2227/105; A01K 2267/01; A61K 2039/505

(57) ABSTRACT

The invention relates to organisms and cells that are engineered to contain exogenous DNA, their use in medicine and the study of disease, methods for production of organisms and cells, and antibodies and antibody chains produced by such organisms and derivatives thereof.

### BACKGROUND

The present invention relates inter alia to rodents and cells that are engineered to contain the exogenous DNA of dogs, their use in medicine and the study of disease, methods for production of rodents and cells, and antibodies and antibody chains produced by such rodents and derivatives thereof.

Insertion of human DNA into rodents has been disclosed in e.g., Murphy et al, Vol 111 no 14, 5153-5158, doi: 10.1073/pnas. 1324022111; MacDonald et al vol. 111 no. 14, 5147-5152, DOI: 10.1073/pnas. 1323896111; and Lee et al, Nature Biotechnology Volume: 32, Pages:356-363 2014 DOI: doi:10.1038/nbt.2825. This approach is designed to make antibody products for human therapeutic use. Insertion of dog DNA into rodents has been disclosed in WO2018189520.

The present invention relates to rodents and cells as well as antibody repertoires, antibodies, and parts of antibodies, such as those produced from rodents comprising dog immunoglobulin DNA, including fully dog antibodies, and use of such antibodies and parts thereof in dogs for prevention and treatment of disease, as well as methods for the manufacture of such rodents, cells, antibodies, antibody chains and repertoires.

### SUMMARY OF THE INVENTION

A rodent or rodent cell having a genome comprising;
i) no more than 8 dog IGHV gene segments, one or more dog IGHD region gene segment(s) and one or more dog IGHJ region gene segment(s), wherein the rodent or rodent cell is capable of expressing the dog IGH gene segments to form an antibody heavy chain, wherein at least one of the IGH V gene segments is selected from the list comprising V3-38, V3-19, V3-35, V3-5, V4-1, V3-41, V3-26 and V1-30.

A rodent or rodent cell having a genome comprising;
i) no more than 17 dog lambda V gene segments and one or more dog IGLJ region gene segment(s), wherein the rodent or rodent cell is capable of expressing the dog lambda gene segments to form an antibody light chain, wherein at least one of the dog lambda gene segments is selected from the list comprising V 1-138, 1-141, 1-55, 1-136, 1-75, 1-48, 8-153, 1-147, 1-125, 1-41, 1-149, 1-58, 1-100, 1-46, 3-21, 1-103, and 1-84.

A rodent or rodent cell having a genome comprising;
i) no more than 5 dog kappa V gene segments and one or more dog IGKJ region gene segment(s); wherein the rodent or rodent cell is capable of expressing the dog kappa gene segments to form an antibody light chain, wherein at least one of the dog kappa gene segments is selected from V 2-8, 2-11, 2-5, 2-4 and 2-7.

A method for producing a rodent or rodent cell disclosed herein, the method comprising inserting into a rodent cell genome:
(i) no more than 8 dog IGHV gene segments, one or more dog IGH D region gene segments and one or more dog IGH J region gene segments, wherein at least one of the IGH V gene segments is selected from the list comprising V3-38, V3-19, V3-35, V3-5, V4-1, V3-41, V3-26 and V1-30; and/or
(ii) no more than 17 dog lambda V gene segments and one or more dog lambda J region gene segments, wherein at least one of the dog lambda V gene segments is selected from the list comprising V 1-138, 1-141, 1-55, 1-136, 1-75, 1-48, 8-153, 1-147, 1-125, 1-41, 1-149, 1-58, 1-100, 1-46, 3-21, 1-103, and 1-84; and/or
(iii) no more than 5 dog kappa V gene segments and one or more dog J region gene segments, wherein at least one of the dog kappa gene segments is selected from V 2-8, 2-11, 2-5, 2-4 and 2-7;
and wherein the rodent or rodent cell is capable of expressing the dog variable region gene segment or segments in combination with a constant region to form an antibody chain.

References herein to the dog variable region gene segments may be references to the dog V region gene segments, the dog D region gene segments and/or the dog J region gene segments as appropriate. In an aspect, the dog variable region gene segments are at least 1 dog V region gene segment, at least one dog D region gene segment and at least one dog J region gene segment.

A method for producing an antibody or antibody chain specific to a desired antigen, the method comprising immunizing a rodent disclosed herein with the desired antigen and recovering the antibody chain or antibody or recovering a cell producing the antibody chain or antibody.

A method for producing an antibody chain or antibody specific to a desired antigen the method comprising immunizing a rodent disclosed herein and then replacing any rodent constant region of the antibody chain or antibody with a dog constant region, suitably by engineering of the nucleic acid encoding the antibody.

References herein to replacing any rodent constant region may mean that in situations where the constant region is a rodent constant region, it is replaced with, for example, a dog constant region and that in situations where the constant region is already, for example, a dog constant region, no such replacement is necessary. This may be the case, for example, at the lambda locus.

A method for producing an antibody, antibody chain or a part thereof, the antibody chain having a dog variable region, the method comprising expressing in a cell a nucleic acid such as DNA encoding the antibody, antibody chain, or a part thereof, wherein the sequence of the nucleic acid encoding the variable region of the antibody chain is obtained from immunising a rodent disclosed herein with an antigen, optionally including the subsequent steps of:
i. purifying and/or isolating the antibody or antibody chain, and
ii. optionally then formulating the antibody or antibody chain into a pharmaceutically acceptable formulation suitable for administration into a dog.

A method of making a pharmaceutical composition, the method comprising producing an antibody, preferably a fully dog antibody, according to a method disclosed herein and further comprising combining the antibody with a pharmaceutically acceptable carrier or other excipient to produce the composition.

A nucleic acid encoding a chimeric antibody chain, the nucleic acid comprising any one of the dog IGHV gene segments selected from the list comprising V3-38, V3-19, V3-35, V3-5, V4-1, V3-41, V3-26 and V1-30 together with a rodent constant region, optionally wherein the dog V gene segment is a sequence that has undergone somatic hypermutation in a rodent.

A nucleic acid encoding a chimeric antibody chain, the nucleic acid comprising any 2, 3, 4, 5, 6, 7, or all 8 of the dog IGHV gene segments selected from the list comprising V3-38, V3-19, V3-35, V3-5, V4-1, V3-41, V3-26 and V1-30 together with a rodent constant region, optionally wherein the dog V gene segment is a sequence that has undergone somatic hypermutation in a rodent.

A nucleic acid encoding a chimeric antibody chain, the nucleic acid comprising any one of the dog lambda V gene segments selected from the list comprising lambda V 1-138, 1-141, 1-55, 1-136, 1-75, 1-48, 8-153, 1-147, 1-125, 1-41, 1-149, 1-58, 1-100, 1-46, 3-21, 1-103 and 1-84 together with a rodent constant region, optionally wherein the dog V gene segment sequence is a sequence that has undergone somatic hypermutation in a rodent.

A nucleic acid encoding a chimeric antibody chain, the nucleic acid comprising any 2, 3, 4, 5, 6, 7, 8, 9, 10, 11, 12, 13, 14, 15, 16 or all 17 of the dog lambda V gene segments selected from the list comprising lambda V 1-138, 1-141, 1-55, 1-136, 1-75, 1-48, 8-153, 1-147, 1-125, 1-41, 1-149, 1-58, 1-100, 1-46, 3-21, 1-103, and 1-84 together with a rodent constant region, optionally wherein the dog V gene segment is a sequence that has undergone somatic hypermutation in a rodent.

A nucleic acid encoding a chimeric antibody chain, the nucleic acid comprising any one of the dog kappa V gene segments selected from the list comprising 2-8, 2-11, 2-5, 2-4 and 2-7 together with a rodent constant region, optionally wherein the dog V gene segment sequence is a sequence that has undergone somatic hypermutation in a rodent.

A nucleic acid encoding a chimeric antibody chain, the nucleic acid comprising any 2, 3, 4, or all 5 of the dog kappa V gene segments selected from the list comprising 2-8, 2-11, 2-5, 2-4 and 2-7 together with a rodent constant region, optionally wherein the dog V gene segment is a sequence that has undergone somatic hypermutation in a rodent.

An antibody chain having a variable region obtained by expression of any one of dog IGH V3-38, V3-19, V3-35, V3-5, V4-1, V3-41, V3-26, and V1-30 in a rodent, in conjunction with a rodent constant region.

An antibody chain having a variable region obtained by expression of any 2, 3, 4, 5, 6, 7, or all 8 of dog IGH V3-38, V3-19, V3-35, V3-5, V4-1, V3-41, V3-26, and V1-30 in a rodent, in conjunction with a rodent constant region.

An antibody chain having a variable region obtained by expression of any one of dog lambda V 1-138, 1-141, 1-55, 1-136, 1-75, 1-48, 8-153, 1-147, 1-125, 1-41, 1-149, 1-58, 1-100, 1-46, 3-21, 1-103, and 1-84 in a rodent, in conjunction with a rodent constant region.

An antibody chain having a variable region obtained by expression of any 2, 3, 4, 5, 6, 7, 8, 9, 10, 11, 12, 13, 14, 15, 16 or all 17 of dog lambda V 1-138, 1-141, 1-55, 1-136, 1-75, 1-48, 8-153, 1-147, 1-125, 1-41, 1-149, 1-58, 1-100, 1-46, 3-21, 1-103, and 1-84 in a rodent, in conjunction with a rodent constant region.

An antibody chain having a variable region obtained by expression of any one of dog kappa V2-8, 2-11, 2-5, 2-4 and 2-7 in a rodent, in conjunction with a rodent constant region.

An antibody chain having a variable region obtained by expression of any 2, 3, 4, or all 5 of dog kappa V2-8, 2-11, 2-5, 2-4 and 2-7 in a rodent, in conjunction with a rodent constant region.

A pharmaceutical composition comprising an antibody chain having a variable region comprising any one of, or any 2 of, 3 of, 4 of, 5 of, 6 of, 7 of, or all 8 of dog IGH V3-38, V3-19, V3-35, V3-5, V4-1, V3-41, V3-26 and V1-30, in conjunction with a pharmaceutically acceptable excipient or carrier.

A pharmaceutical composition comprising an antibody chain having a variable region comprising any one of, or any 2 of, 3 of, 4 of, 5 of, 6 of, 7 of, 8 of, 9 of, 10 of, 11 of, 12 of, 13 of, 14 of, 15 of, 16 of, or all 17 of dog lambda V 1-138, 1-141, 1-55, 1-136, 1-75, 1-48, 8-153, 1-147, 1-125, 1-41, 1-149, 1-58, 1-100, 1-46, 3-21, 1-103 and 1-84 in conjunction with a pharmaceutically acceptable excipient or carrier.

A pharmaceutical composition comprising an antibody chain having a variable region comprising any one of, or any 2 of, 3 of, 4 of, or all 5 of dog kappa V2-8, 2-11, 2-5, 2-4 and 2-7 in conjunction with a pharmaceutically acceptable excipient or carrier.

A pharmaceutical composition comprising an antibody, the antibody having:
(i) a heavy chain having a variable region obtained by expression of any one of dog IGH V3-38, V3-19, V3-35, V3-5, V4-1, V3-41, V3-26 and V1-30, and either
(ii) a lambda light chain obtained by expression of any one of dog lambda V 1-138, 1-141, 1-55, 1-136, 1-75, 1-48, 8-153, 1-147, 1-125, 1-41, 1-149, 1-58, 1-100, 1-46, 3-21, 1-103 and 1-84; or
(iii) a kappa light chain obtained by expression of any one of dog kappa V2-8, 2-11, 2-5, 2-4 and 2-7, the antibody being formulated in conjunction with a pharmaceutically acceptable excipient or carrier.

An antibody heavy chain repertoire, the repertoire comprising antibody heavy chains having dog IgHV gene segments from no more than 8 different dog IGH V gene segments, wherein at least one of the IGH V gene segments is selected from the list comprising V3-38, V3-19, V3-35, V3-5, V4-1, V3-41, V3-26 and V1-30;
wherein optionally the repertoire comprises:
   (a) antibody heavy chains having dog IgHV gene segments expressed from:
      (i) V3-38, optionally with up to 7 other dog IGH V gene segments;
      (ii) V3-38 and V3-19, optionally with up to 6 other dog IGH V gene segments;
      (iii) V3-38, V3-19 and V3-35, optionally with up to 5 other dog IGH V gene segments;
      (iv) V3-38, V3-19, V3-35, and V3-5, optionally with up to 4 other dog IGH V gene segments;
      (v) V3-38, V3-19, V3-35, V3-5, V4-1, optionally with up to 3 other dog IGH V gene segments;
      (vi) V3-38, V3-19, V3-35, V3-5, V4-1, V3-41, optionally with up to 2 other dog IGH V gene segments;
      (vii) V3-38, V3-19, V3-35, V3-5, V4-1, V3-41, V3-26, optionally with up to 1 other dog IGH V gene segment; and
      (viii) V3-38, V3-19, V3-35, V3-5, V4-1, V3-41, V3-26 and V1-30; and/or
   (b) antibody heavy chains expressed from IGHV gene segments of dog IGHV families 1, 3 and 4.

Families of genes, such as IGHV family 1, are well-known terms in the art. In an aspect, reference to families of genes, such as IGHV family 1, refers to the notation of the IMGT Repertoire (ImMunoGeneTics), which is available (as of 19 May 2022) at: https://www.imgt.org/IMGTrepertoire/index.php?section=LocusGenes&repertoire=genetable&species=dog&group=IGH V.

An antibody heavy chain repertoire, the repertoire comprising antibody heavy chains having dog IgHV gene segments from no more than 8 different dog IGH V gene segments, wherein at least one of the IGH V gene segments is selected from the list comprising V3-38, V3-19, V3-35, V3-5, V4-1, V3-41, V3-26 and V1-30 wherein optionally the repertoire comprises:
(a) antibody heavy chains having dog IgHV gene segments expressed from:
   (i) V3-38, optionally with up to 7 other dog IGH V gene segments;
   (ii) V3-38 and V3-19, optionally with up to 6 other dog IGH V gene segments;
   (iii) V3-38, V3-19 and V3-35, optionally with up to 5 other dog IGH V gene segments;
   (iv) V3-38, V3-19, V3-35, and V3-5, optionally with up to 4 other dog IGH V gene segments;
   (v) V3-38, V3-19, V3-35, V3-5, V4-1, optionally with up to 3 other dog IGH V gene segments;
   (vi) V3-38, V3-19, V3-35, V3-5, V4-1, V3-41, optionally with up to 2 other dog IGH V gene segments;
   (vii) V3-38, V3-19, V3-35, V3-5, V4-1, V3-41, V3-26, optionally with up to 1 other dog IGH V gene segment; or
   (viii) V3-38, V3-19, V3-35, V3-5, V4-1, V3-41, V3-26 and V1-30; and/or
(b) antibody heavy chains expressed from IGHV gene segments of dog IGHV families 1, 3 and 4.

An antibody heavy chain repertoire, the repertoire comprising antibody heavy chains having dog IgHV gene segments from no more than 8 different dog IGH V gene segments, wherein at least one of the IGH V gene segments is selected from the list comprising V3-38, V3-19, V3-35, V3-5, V4-1, V3-41, V3-26 and V1-30 wherein optionally the repertoire comprises:
(a) antibody heavy chains having dog IgHV gene segments expressed from:
   (i) V3-38, optionally with up to 7 other dog IGH V gene segments, further optionally wherein 1, 2, 3, 4, 5, 6 or all 7 of the other dog IGH V gene segments are selected from the same list;
   (ii) V3-38 and V3-19, optionally with up to 6 other dog IGH V gene segments, further optionally wherein 1, 2, 3, 4, 5, or all 6 of the other dog IGH V gene segments are selected from the same list;
   (iii) V3-38, V3-19 and V3-35, optionally with up to 5 other dog IGH V gene segments, further optionally wherein 1, 2, 3, 4, or all 5 of the other dog IGH V gene segments are selected from the same list;
   (iv) V3-38, V3-19, V3-35, and V3-5, optionally with up to 4 other dog IGH V gene segments, further optionally wherein 1, 2, 3, or all 4 of the other dog IGH V gene segments are selected from the same list;
   (v) V3-38, V3-19, V3-35, V3-5, V4-1, optionally with up to 3 other dog IGH V gene segments, further optionally wherein 1, 2, or all 3 of the other dog IGH V gene segments are selected from the same list;
   (vi) V3-38, V3-19, V3-35, V3-5, V4-1, V3-41, optionally with up to 2 other dog IGH V gene segments, further optionally wherein 1, or both of the other dog IGH V gene segments are selected from the same list;
   (vii) V3-38, V3-19, V3-35, V3-5, V4-1, V3-41, V3-26, optionally with up to 1 other dog IGH V gene segment, further optionally wherein the up to 1 other dog IGH V gene segment is selected from the same list; and/or
   (viii) V3-38, V3-19, V3-35, V3-5, V4-1, V3-41, V3-26 and V1-30; and/or
(b) antibody heavy chains expressed from IGHV gene segments of dog IGHV families 1, 3 and 4.

An antibody heavy chain repertoire, the repertoire comprising antibody heavy chains having dog IGHV gene segments from no more than 8 different dog IGH V gene segments, where 1 IGHV segment must be IGHV4-1, 1 IGHV segment must be IGHV1-30, and at least one of the remaining IGH V segments is selected from the list comprising V3-38, V3-19, V3-35, V3-5, V3-41, and V3-26.

An antibody lambda light chain repertoire, the repertoire comprising antibody light chains having dog lambda V gene segments from no more than 17 different lambda V gene segments, wherein at least one of the V gene segments is selected from the list comprising 1-138, 1-141, 1-55, 1-136, 1-75, 1-48, 8-153, 1-147, 1-125, 1-41, 1-149, 1-58, 1-100, 1-46, 3-21, 1-103 and 1-84, wherein optionally the repertoire comprises:
(a) antibody lambda light chains having dog lambda V gene segments including
   (i) 1-138, optionally with up to 16 other dog lambda V gene segments;
   (ii) 1-138, 1-141 optionally with up to 15 other dog lambda V gene segments;
   (iii) 1-138, 1-141, 1-55, optionally with up to 14 other dog lambda V gene segments;
   (iv) 1-138, 1-141, 1-55, 1-136, optionally with up to 13 other dog lambda V gene segments;
   (v) 1-138, 1-141, 1-55, 1-136, 1-75, optionally with up to 12 other dog lambda V gene segments;
   (vi) 1-138, 1-141, 1-55, 1-136, 1-75, 1-48, optionally with up to 11 other dog lambda V gene segments;
   (vii) 1-138, 1-141, 1-55, 1-136, 1-75, 1-48, 8-153, optionally with up to 10 other dog lambda V gene segments; and/or
(b) antibody lambda light chains expressed from lambda V gene segments of dog lambda families 1, 2, 3, 4, 5 and 8.

An antibody lambda light chain repertoire, the repertoire comprising antibody light chains having dog lambda V gene segments from no more than 17 different lambda V gene segments, wherein at least one of the V gene segments is selected from the list comprising 1-138, 1-141, 1-55, 1-136, 1-75, 1-48, 8-153, 1-147, 1-125, 1-41, 1-149, 1-58, 1-100, 1-46, 3-21, 1-103 and 1-84, wherein optionally the repertoire comprises:
(a) antibody lambda light chains having dog lambda V gene segments including
   (i) 1-138, optionally with up to 16 other dog lambda V gene segments;
   (ii) 1-138, 1-141 optionally with up to 15 other dog lambda V gene segments;
   (iii) 1-138, 1-141, 1-55, optionally with up to 14 other dog lambda V gene segments;
   (iv) 1-138, 1-141, 1-55, 1-136, optionally with up to 13 other dog lambda V gene segments;
   (v) 1-138, 1-141, 1-55, 1-136, 1-75, optionally with up to 12 other dog lambda V gene segments;
   (vi) 1-138, 1-141, 1-55, 1-136, 1-75, 1-48, optionally with up to 11 other dog lambda V gene segments;
   (vii) 1-138, 1-141, 1-55, 1-136, 1-75, 1-48, 8-153, optionally with up to 10 other dog lambda V gene segments; and/or
(b) antibody lambda light chains expressed from lambda V gene segments of dog lambda families 1, 2, 3, 4, 5 and 8.

An antibody lambda light chain repertoire, the repertoire comprising antibody light chains having dog lambda V gene segments from no more than 17 different lambda V gene segments, wherein at least one of the V gene segments is selected from the list comprising 1-138, 1-141, 1-55, 1-136, 1-75, 1-48, 8-153, 1-147, 1-125, 1-41, 1-149, 1-58, 1-100, 1-46, 3-21, 1-103 and 1-84, wherein optionally the repertoire comprises:
(a) antibody lambda light chains having dog lambda V gene segments including
   (i) 1-138, optionally with up to 16 other dog lambda V gene segments, further optionally wherein 1, 2, 3, 4, 5, 6, 7, 8, 9, 10, 11, 12, 13, 14, 15, or all 16 of the other dog lambda V gene segments are selected from the same list;
   (ii) 1-138, 1-141 optionally with up to 15 other dog lambda V gene segments, further optionally wherein 1, 2, 3, 4, 5, 6, 7, 8, 9, 10, 11, 12, 13, 14, or all 15 of the other dog lambda V gene segments are selected from the same list;
   (iii) 1-138, 1-141, 1-55, optionally with up to 14 other dog lambda V gene segments, further optionally wherein 1, 2, 3, 4, 5, 6, 7, 8, 9, 10, 11, 12, 13, or all 14 of the other dog lambda V gene segments are selected from the same list;
   (iv) 1-138, 1-141, 1-55, 1-136, optionally with up to 13 other dog lambda V gene segments, further optionally wherein 1, 2, 3, 4, 5, 6, 7, 8, 9, 10, 11, 12, or all 13 of the other dog lambda V gene segments are selected from the same list;
   (v) 1-138, 1-141, 1-55, 1-136, 1-75, optionally with up to 12 other dog lambda V gene segments, further optionally wherein 1, 2, 3, 4, 5, 6, 7, 8, 9, 10, 11, or all 12 of the other dog lambda V gene segments are selected from the same list;
   (vi) 1-138, 1-141, 1-55, 1-136, 1-75, 1-48, optionally with up to 11 other dog lambda V gene segments, further optionally wherein 1, 2, 3, 4, 5, 6, 7, 8, 9, 10, or all 11 of the other dog lambda V gene segments are selected from the same list; or
   (vii) 1-138, 1-141, 1-55, 1-136, 1-75, 1-48, 8-153, optionally with up to 10 other dog lambda V gene segments, further optionally wherein 1, 2, 3, 4, 5, 6, 7, 8, 9, or all 10 of the other dog lambda V gene segments are selected from the same list; and/or
(b) antibody lambda light chains expressed from lambda V gene segments of dog lambda families 1, 2, 3, 4, 5 and 8.

An antibody kappa light chain repertoire, the repertoire comprising antibody kappa light chains having dog kappa V gene segments from no more than 5 different kappa V gene segments, wherein at least one of the V gene segments is selected from the list comprising V2-8, 2-11, 2-5, 2-4 and 2-7, optionally wherein the repertoire comprises:
(a) antibody kappa light chains having dog kappa V gene segments including
   (i) 2-8, optionally with up to 4 other dog lambda V gene segments;
   (ii) 2-8, 2-11 optionally with up to 3 other dog lambda V gene segments;
   (iii) 2-8, 2-11, 2-5 optionally with up to 2 other dog lambda V gene segments;
   (iv) 2-8, 2-11, 2-5, 2-4 optionally with up to 1 other dog lambda V gene segment; or
   (v) 2-8, 2-11, 2-5, 2-4 and 2-7; and/or
(b) antibody kappa light chains expressed from kappa V gene segments of dog kappa V families 2, 3 and 4.

An antibody kappa light chain repertoire, the repertoire comprising antibody kappa light chains having dog kappa V gene segments from no more than 5 different kappa V gene segments, wherein at least one of the V gene segments is selected from the list comprising V2-8, 2-11, 2-5, 2-4 and 2-7, optionally wherein the repertoire comprises:
(a) antibody kappa light chains having dog kappa V gene segments including
   (i) 2-8, optionally with up to 4 other dog kappa V gene segments;
   (ii) 2-8, 2-11 optionally with up to 3 other dog kappa V gene segments;
   (iii) 2-8, 2-11, 2-5 optionally with up to 2 other dog kappa V gene segments;
   (iv) 2-8, 2-11, 2-5, 2-4 optionally with up to 1 other dog kappa V gene segment; or
   (v) 2-8, 2-11, 2-5, 2-4 and 2-7; and/or
(b) antibody kappa light chains expressed from kappa V gene segments of dog kappa V families 2, 3 and 4.

An antibody kappa light chain repertoire, the repertoire comprising antibody kappa light chains having dog kappa V gene segments from no more than 5 different kappa V gene segments, wherein at least one of the V gene segments is selected from the list comprising V2-8, 2-11, 2-5, 2-4 and 2-7, optionally wherein the repertoire comprises:
(a) antibody kappa light chains having dog kappa V gene segments including
   (i) 2-8, optionally with up to 4 other dog kappa V gene segments, further optionally wherein 1, 2, 3, or all 4 of the other dog kappa V gene segments are selected from the same list;
   (ii) 2-8, 2-11 optionally with up to 3 other dog kappa V gene segments, further optionally wherein 1, 2, or all 3 of the other dog kappa V gene segments are selected from the same list;
   (iii) 2-8, 2-11, 2-5 optionally with up to 2 other dog kappa V gene segments, further optionally wherein 1 or both of the other dog kappa V gene segments are selected from the same list;
   (iv) 2-8, 2-11, 2-5, 2-4 optionally with up to 1 other dog kappa V gene segment, further optionally wherein the up to 1 other dog kappa gene segment is selected from the same list; or
   (v) 2-8, 2-11, 2-5, 2-4 and 2-7; and/or
(b) antibody kappa light chains expressed from kappa V gene segments of dog kappa V families 2, 3 and 4.

An antibody kappa light chain repertoire, the repertoire comprising antibody kappa light chains having dog kappa V gene segments from no more than 5 different kappa V gene segments, wherein one of the V gene segments must be IGKV3-18, one must be IGKV4-15, and at least one of the V gene segments is selected from the list comprising V2-8, 2-11, 2-5, 2-4 and 2-7.

An isolated antibody or antibody chain, or part thereof, or a nucleic acid such as DNA encoding an antibody chain or a part thereof, which has been obtained or is obtainable from a rodent or rodent cell described herein, or from a repertoire described herein, such as a fully dog antibody chain or fully dog antibody, or a pharmaceutical composition comprising the same.

An antibody or antibody or chain, or part thereof, or a nucleic acid such as DNA encoding an antibody chain or a part thereof, which has been obtained or is obtainable from a rodent or rodent cell as described herein, or from a repertoire described herein, such as a fully dog antibody chain or fully dog antibody, or a pharmaceutical composition comprising the same, for use in treatment or prevention of disease in a dog in need thereof.

A method of treatment of a dog, the method comprising delivery of an antibody or antibody chain or part thereof to a dog in need thereof, the antibody being obtained or obtainable from a rodent or rodent cell or repertoire as disclosed herein.

### FIGURES AND TABLES

Table 1a—Analysis of healthy dog PBMC, listing the most commonly observed heavy and light chain pairings

Table 1b—Analysis of healthy dog PBMC, listing the most commonly observed heavy chains

Table 1c—Analysis of healthy dog PBMC, listing the most commonly observed light chains

Table 2—primer sequences

Table 3—Analysis of healthy dog PBMC, listing the light chains ranked by number of different heavy chain pairings

Table 4—List of preferred V gene sequences

FIG. 1—Graphical representation of common antibody pairings in dogs

### DETAILED DESCRIPTION

The present invention relates to rodents which comprise dog immunoglobulin gene segments. Prior art rodents, both those containing human IG DNA and dog DNA, generally look to insert as many V gene segments as possible to optimize the range of antibodies produced. However, this requires a considerable amount of genome engineering effort to achieve a complete insertion of the ˜1 MB of human DNA. In the present disclosure we have analyzed the profile of antibodies made in beagles and shown that only a few V gene segments contribute disproportionately to the antibody pool. For example, the IGH V3-38 gene segment is found in 33% of heavy chains of antibodies. Therefore, it is not necessary to insert all of the V gene segments in order to create an antibody population that will provide a suitable repertoire for the identification and selection of lead antibody candidates. This streamlines the genetic engineering needed to make a rodent model for antibody production.

If a large portion (such as all) of the dog Ig locus is inserted into the rodent, some V gene segments preferentially used in dogs might be less frequently expressed in the rodent, due to their distance upstream from the (rodent) constant region. This could reduce the effectiveness of such a rodent to produce therapeutically relevant antibodies. Therefore, another advantage of the present invention is that preferred V gene segments can be inserted closer to the constant region than their natural position in dogs, and therefore these preferred V gene segments are likely to be used more often in the rodent than if a larger portion of the dog Ig locus is inserted, thus more faithfully representing the natural repertoire of a dog.

The dog V gene segments chosen to be inserted into a rodent to create an antibody population could be based on observed frequency, on the number of different pairings (between heavy dog V gene segments and light dog V gene segments), or a combination of the two. The highest observed frequency dog V gene segments are preferred. Preferred heavy or light dog V gene segments are those comprised within antibody chains which pair with the highest number of different light or heavy dog V antibody chains respectively (antibody chains that comprise different dog V gene segments). The invention therefore relates to:

A rodent or rodent cell having a genome comprising;
i) no more than 8 dog IGHV gene segments, one or more dog D region gene segments and one or more dog J region gene segments,
ii) wherein the rodent or rodent cell is capable of expressing the dog gene segments to form an antibody heavy chain, and
iii) wherein at least one of the IGH V gene segments is selected from the list comprising V3-38, V3-19, V3-35, V3-5, V4-1, V3-41, V3-26 and V1-30.

Table 1b lists the frequency of observed dog heavy chains V gene segments. One or more of the 8 preferred gene segments may be combined in the rodent genome with any other dog V gene segment or segments. It is not necessary to limit only to the preferred 8 V gene segments, but at least one must be present. A preferred gene segment to be present is IGH V3-38. An alternative gene segment that is preferred is V3-19. These 2 sequences together are together found in over 50% of the dog antibody heavy chains.

A further embodiment disclosed herein is a rodent or rodent cell disclosed herein comprising dog IGH
(i) V3-38, optionally with up to 7 other dog IGH V gene segments;
(ii) V3-38 and V3-19, optionally with up to 6 other dog IGH V gene segments;
(iii) V3-38, V3-19 and V3-35, optionally with up to 5 other dog IGH V gene segments;
(iv) V3-38, V3-19, V3-35, and V3-5, optionally with up to 4 other dog IGH V gene segments;
(v) V3-38, V3-19, V3-35, V3-5, V4-1, optionally with up to 3 other dog IGH V gene segments;
(vi) V3-38, V3-19, V3-35, V3-5, V4-1, V3-41, optionally with up to 2 other dog IGH V gene segments;
(vii) v3-38, V3-19, V3-35, V3-5, V4-1, V3-41, V3-26, optionally with up to 1 other dog IGH V gene segment;
(viii) V3-38, V3-19, V3-35, V3-5, V4-1, V3-41, V3-26 and V1-30.

A further embodiment disclosed herein is a rodent or rodent cell disclosed herein comprising dog IGH
(i) V3-38, optionally with up to 7 other dog IGH V gene segments;
(ii) V3-38 and V3-19, optionally with up to 6 other dog IGH V gene segments;
(iii) V3-38, V3-19 and V3-35, optionally with up to 5 other dog IGH V gene segments;
(iv) V3-38, V3-19, V3-35, and V3-5, optionally with up to 4 other dog IGH V gene segments;
(v) V3-38, V3-19, V3-35, V3-5, V4-1, optionally with up to 3 other dog IGH V gene segments;
(vi) V3-38, V3-19, V3-35, V3-5, V4-1, V3-41, optionally with up to 2 other dog IGH V gene segments;
(vii) v3-38, V3-19, V3-35, V3-5, V4-1, V3-41, V3-26, optionally with up to 1 other dog IGH V gene segment; or
(viii) V3-38, V3-19, V3-35, V3-5, V4-1, V3-41, V3-26 and V1-30.

A further embodiment disclosed herein is a rodent or rodent cell comprising no more than 7, no more than 6, no more than 5, no more than 4, no more than 3, no more than two or only 1 of the dog IGH V gene segment(s) V3-38, V3-19, V3-35, V3-5, V4-1, V3-41, V3-26 and V1-30.

A further embodiment disclosed herein is a rodent or rodent cell having only 1 dog IGH V gene segment as listed above, and only 1 dog IGH D gene segment and only 1 dog IGH J gene segment, forming a “common heavy chain”.

In an aspect, a common heavy chain approach can be used in a method to generate bispecific antibodies.

A further embodiment disclosed herein is a rodent or rodent cell comprising at least 2 IgH gene segments, such as at least 3, at least 4, at least 5, at least 6, at least 7 or all 8 of the dog IGH V gene segments V3-38, V3-19, V3-35, V3-5, V4-1, V3-41, V3-26 and V1-30. The rodent or rodent cell may comprise 1, 2, 3, 4, 5, 6, 7 or 8 of the dog IGH V gene segments V3-38, V3-19, V3-35, V3-5, V4-1, V3-41, V3-26 and V1-30.

In one embodiment of any aspect of the invention, the total number of dog V gene segments inserted at each locus (heavy, lambda and/or kappa) is 8 or fewer. In one embodiment of any aspect of the invention, the total number of dog V gene segments inserted at each locus (heavy, lambda and/or kappa) is 17 or fewer. In one embodiment of any aspect of the invention, the total number of dog V gene segments inserted at each locus (heavy, lambda and/or kappa) is 5 or fewer. In one embodiment of any aspect of the invention, the total number of dog V gene segments inserted at each locus (heavy, lambda and/or kappa) is 20 or fewer. In one embodiment of any aspect of the invention, the total number of dog V gene segments inserted at each locus (heavy, lambda and/or kappa) is 35 or fewer. In one embodiment of any aspect of the invention, the total number of dog V gene segments inserted at each locus (heavy, lambda and/or kappa) is 50 or fewer.

It is preferred that the rodent comprises at least one gene segment from each of dog IGH V gene families 1, 3 and 4, there being no more than 8 dog IGHV gene segments in the rodent or rodent cell genome.

In an embodiment, a subset of the total number of preferred dog V gene segments is inserted. 1, 2, 3, 4, 5, 6, 7, or 8 of the preferred dog IGH V gene segments may be inserted. 1, 2, 3, 4, 5, 6, 7, 8, 9, 10, 11, 12, 13, 14, 15, 16, or 17 of the preferred dog IGL V gene segments may be inserted. 1, 2, 3, 4, or 5 of the preferred dog IGK V gene segments may be inserted. The total number of dog V gene segments inserted at each locus may be limited, as disclosed herein, so that in combination, for example, the invention contemplates insertion of 20 dog IGH V gene segments wherein 6 of the inserted dog IGH V gene segments are preferred dog IGH V gene segments.

In a further embodiment, the preferred dog V gene segments are inserted together with other dog V gene segments, and/or other regions of the dog Ig locus. In particular, the chromosomal regions surrounding the preferred dog V gene may be inserted together therewith.

For example, an inserted dog V gene segment may be inserted as part of a single bacterial artificial chromosome (BAC). In an aspect, each BAC may contain 3-5 dog V gene segments, including one of the preferred dog V gene segments. In an aspect, reference to a BAC, such as BAC CHORI-82-406K8 is a reference to the CHORI-82 BAC Library, available (as of 19 May 2022) at: https:/bacpacresources.org/hbrary.php?id=253 and described in Osoegawa et al., 1998 (An Improved Approach for Construction of Bacterial Artificial Chromosome Libraries. Genomics, 52(1), 1-8. https://doi.org/10.1006/geno.1998.5423).

In an aspect, inserting a dog IGH V gene segment may involve insertion of some or all of BAC CHORI-82-406K8. In an aspect, inserting a dog IGH V gene segment may involve insertion of some or all of BAC CHORI-82-406K8 and some or all of BAC CHORI-82-40L18. In an aspect, inserting a dog IGH V gene segment may involve insertion of some or all of BAC CHORI-82-406K8, some or all of BAC CHORI-82-40L18 and some or all of BAC CHORI-82-61C7.

In an aspect, inserting a dog IGL V gene segment may involve insertion of some or all of BAC CHORI-82-448M7. In an aspect, inserting a dog IGL V gene segment may involve insertion of some or all of BAC CHORI-82-448M7 and some or all of BAC CHORI-82-101P14. In an aspect, inserting a dog IGL V gene segment may involve insertion of some or all of BAC CHORI-82-448M7, some or all of BAC CHORI-82-101P14 and some or all of BAC CHORI-82-265F20.

In an aspect, inserting a dog IGK V gene segment may involve insertion of some or all of BAC CHORI-82-450E2.

In an embodiment, the inserted dog gene segments may be inserted as part of an array or cluster of dog gene segments, optionally embedded in rodent or dog noncoding regulatory or scaffold sequences.

In an embodiment, the inserted dog gene segments may be inserted as part of a mini-locus.

Alternative methods of inserting gene segments into a rodent are described in US20170306352, which is incorporated by reference.

In an embodiment, the inserted dog V gene segments are inserted as part of a concatemer. In an aspect, the concatemer is a series of preferred dog V gene segments, optionally with dog or rodent regulatory sequences associated with the dog V gene segments.

The invention further relates to:

A rodent or rodent cell having a genome comprising;
i) no more than 17 dog lambda V gene segments, and one or more dog J region gene segments;
ii) wherein the rodent or rodent cell is capable of expressing the dog lambda gene segments to form an antibody light chain,
iii) wherein at least one of the dog lambda gene segments is selected from 1-138, 1-141, 1-55, 1-136, 1-75, 1-48, 8-153, 1-147, 1-125, 1-41, 1-149, 1-58, 1-100, 1-46, 3-21, 1-103, and 1-84.

Table 1c lists the frequency of observed dog light chain V gene segments. One or more of the 17 preferred lambda gene segments may be combined in the rodent genome with any other V gene segment or segments. It is not necessary to limit only to the preferred 17 V gene segments, but at least one must be present. A preferred gene segment to be present is IGH 1-138. An alternative gene segment that is preferred is 1-141. An alternative gene segment that is preferred is 1-55. An alternative gene segment that is preferred is 1-136. These 4 sequences together are together found in over 33% of the lambda antibody chains.

A further embodiment disclosed herein is a rodent or rodent cell comprising dog lambda V
(i) 1-138, optionally with up to 16 other dog lambda V gene segments;
(ii) 1-138, 1-141 optionally with up to 15 other dog lambda V gene segments;
(iii) 1-138, 1-141, 1-55, optionally with up to 14 other dog lambda V gene segments;
(iv) 1-138, 1-141, 1-55, 1-136, optionally with up to 13 other dog lambda V gene segments;
(v) 1-138, 1-141, 1-55, 1-136, 1-75, optionally with up to 12 other dog lambda V gene segments;
(vii) 1-138, 1-141, 1-55, 1-136, 1-75, 1-48, optionally with up to 11 other dog lambda V gene segments;
(viii) 1-138, 1-141, 1-55, 1-136, 1-75, 1-48, 8-153, optionally with up to 10 other dog lambda V gene segments.

A further embodiment disclosed herein is a rodent or rodent cell comprising no more than 16, no more than 15, no more than 14, no more than 13, no more than 12, no more than 11, no more than 10, no more than 9, no more than 8, no more than 7, no more than 6, no more than 5, no more than 4, no more than 3, no more than 2 or only 1 V gene segment selected from the list of 1-138, 1-141, 1-55, 1-136, 1-75, 1-48, 8-153, 1-147, 1-125, 1-41, 1-149, 1-58, 1-100, 1-46, 3-21, 1-103, and 1-84.

A further embodiment disclosed herein is a rodent or rodent cell having only 1 dog lambda V gene segment, as listed above, and only 1 dog lambda J gene segment, forming a “common light chain”.

A further embodiment disclosed herein is a rodent or rodent cell comprising at least 2 dog lambda V gene segments, such as at least 3, at least 4, at least 5, at least 6, at least 7, at least 8, at least 9, at least 10, at least 11, at least 12, at least 13, at least 14, at least 15 or at least 16 dog lambda V gene segments selected from 1-138, 1-141, 1-55, 1-136, 1-75, 1-48, 8-153, 1-147, 1-125, 1-41, 1-149, 1-58, 1-100, 1-46, 3-21, 1-103 and 1-84. The rodent or rodent cell may comprise 1, 2, 3, 4, 5, 6, 7, 8, 9, 10, 11, 12, 13, 14, 15, 16 or 17 of the dog lambda gene segments 1-138, 1-141, 1-55, 1-136, 1-75, 1-48, 8-153, 1-147, 1-125, 1-41, 1-149, 1-58, 1-100, 1-46, 3-21, 1-103, and 1-84.

It is preferred that the rodent comprises at least one lambda V gene segment from each of the dog lambda V gene segment families V1, 2, 3, 4, 5 and 8, there being no more than 17 dog lambda V gene segments in the genome.

The invention further relates to:

A rodent or rodent cell having a genome comprising;
i) no more than 5 dog kappa V gene segments and one or more dog J region gene segment(s);
ii) wherein the rodent or rodent cell is capable of expressing the dog kappa gene segments to form an antibody light chain,
iii) wherein at least one of the dog kappa gene segments is selected from 2-8, 2-11, 2-5, 2-4 and 2-7.

Table 1c lists the frequency of observed dog light chain V gene segments. One or more of the 5 preferred kappa gene segments may be combined in the rodent genome with any other V gene segments. It is not necessary to limit only to the preferred 5 kappa V gene segments, but at least one must be present. A preferred gene segment to be present is V2-8, which is seen in 28% of all kappa chain antibodies. An alternative gene segment that is preferred is V2-11. An alternative gene segment that is preferred is V2-5. These 3 sequences together are together found in over 63% of the lambda antibody chains.

A further embodiment disclosed herein is a rodent or rodent cell comprising dog kappa V
(i) 2-8, optionally with up to 4 other dog lambda V gene segments;
(ii) 2-8, 2-11 optionally with up to 3 other dog lambda V gene segments;
(iii) 2-8, 2-11, 2-5 optionally with up to 2 other dog lambda V gene segments;
(iv) 2-8, 2-11, 2-5, 2-4 optionally with up to 1 other dog lambda V gene segments; or
(v) 2-8, 2-11, 2-5, 2-4 and 2-7

A further embodiment disclosed herein is a rodent or rodent cell comprising dog kappa V
(i) 2-8, optionally with up to 4 other dog kappa V gene segments;
(ii) 2-8, 2-11 optionally with up to 3 other dog kappa V gene segments;
(iii) 2-8, 2-11, 2-5 optionally with up to 2 other dog kappa V gene segments;
(iv) 2-8, 2-11, 2-5, 2-4 optionally with up to 1 other dog kappa V gene segments; or
(v) 2-8, 2-11, 2-5, 2-4 and 2-7.

A further embodiment disclosed herein is a rodent or rodent cell comprising no more than 4, no more than 3, no more than 2, or only 1 dog kappa V gene segment selected from V2-8, 2-11, 2-5, 2-4 and 2-7.

A further embodiment disclosed herein is a rodent or rodent cell having only 1 dog kappa V gene segment, as listed above, and only 1 dog kappa J gene segment, forming a “common light chain”.

A further embodiment disclosed herein is a rodent or rodent cell comprising at least 2 dog kappa V gene segments, such as at least 3, at least 4, or 5 dog kappa V gene segments selected from V 2-8, 2-11, 2-5, 2-4 and 2-7. A further embodiment disclosed herein is a rodent or rodent cell comprising 1, 2, 3, 4 or 5 dog kappa V gene segments selected from V 2-8, 2-11, 2-5, 2-4 and 2-7.

It is preferred that the rodent comprises a kappa V gene segment from each of the kappa V gene segment families 3 and 4, suitably including IGKV3-18 and IGKV4-15, with there being no more than 5 dog kappa V gene segments in the genome.

In another preferred aspect the rodent genome comprises dog DNA encoding any one, or more, of the following heavy and light chain pairs, which together form over 20% of the observed antibody chain pairings:

| | |
|---|---|
| IGHV3-38cf | IGLV1-138cf |
| IGHV3-38cf | IGLV1-55cf |
| IGHV3-19cf | IGLV1-138cf |
| IGHV3-38cf | IGLV1-48cf |
| IGHV3-38cf | IGLV1-125cf |
| IGHV3-38cf | IGLV1-75cf |
| IGHV3-19cf | IGLV1-136cf |
| IGHV3-19cf | IGLV8-153cf |
| IGHV3-38cf | IGLV1-136cf |
| IGHV3-38cf | IGKV2-8cf |
| IGHV3-19cf | IGLV1-141cf |
| IGHV3-38cf | IGLV1-141cf |
| IGHV3-19cf | IGLV1-147cf |
| IGHV3-19cf | IGLV1-48cf |
| IGHV3-38cf | IGLV8-153cf |
| IGHV3-38cf | IGLV1-58cf |
| IGHV3-19cf | IGLV1-149cf |
| IGHV3-35cf | IGLV1-75cf |
| IGHV3-38cf | IGLV1-100cf |
| IGHV3-38cf | IGLV1-41cf |
| IGHV3-38cf | IGLV1-147cf |
| IGHV4-1cf | IGLV1-141cf |
| IGHV3-19cf | IGKV2-8cf |
| IGHV3-19cf | IGLV1-55cf |
| IGHV3-38cf | IGLV1-46cf |
| IGHV3-38cf | IGLV3-3cf |

The inserted DNA in which the number of V gene segments is limited may be referred to herein as the constrained insertion or the constrained locus. It will be appreciated that some embodiments of the invention allow for an unconstrained inserted number of heavy chain V gene segments (when the light chain is constrained), or vice versa allow for an unconstrained inserted number of light chain V gene segments when the heavy chain is constrained. 1, 2 or all 3 dog insertions may be constrained.

The number of dog V gene segments at the constrained locus suitably provides at least 80% coverage of the observed antibody sequences, such as at least 85%, 90%, 95%, 97%, 98%, or 99% coverage, optionally wherein coverage of the observed antibody sequences is less than 100%.

The number of dog V gene segments at the constrained locus, which may be a single dog V gene segment, suitably provides at least 10% coverage of the observed antibody sequences, such as at least 20%, 30%, 40%, 50%, 60% or 70% coverage, optionally wherein coverage of the observed antibody sequences is less than 100%.

The number of dog V gene segments at the constrained locus suitably provides at most 80% coverage of the observed antibody sequences, such as at most 85%, 90%, 95%, 97%, 98%, or 99% coverage.

The number of dog V gene segments at the constrained locus, which may be a single dog V gene segment, suitably provides around 33% coverage of the observed antibody sequences, or around 40%, around 50%, around 60% or around 70% coverage.

For example, the rodent or cell genome may comprise the constrained dog heavy chain insertion as disclosed herein in combination with a constrained dog lambda light chain insertion as disclosed herein, and/or with a constrained dog light chain kappa chain insertion as disclosed herein.

The rodent or rodent cell genome of the invention may comprise one or more dog IGHV gene segments as described herein, as a constrained insertion, but comprise no light chain dog DNA, or may comprise one or more dog IGLV gene segments as described herein as a constrained insertion but no heavy chain dog DNA.

Where an immunoglobulin locus of a rodent or rodent cell genome is not constrained by the number of V gene segments, it may have any number, including all, of the dog V gene segments. The rodent or cell genome may therefore comprise an inserted dog heavy chain as disclosed herein in combination with any light chain insertion—such as a fully dog light chain, or a dog light chain insertion having more than 17 dog V lambda gene segments and/or more than 5 dog kappa V gene segments. Alternatively, the rodent or cell genome may comprise the dog light chain insertion as disclosed herein in combination with any heavy chain—such as a fully dog heavy chain, or a dog heavy chain insertion having more than 8 dog V gene segments.

For example, where only the heavy chain insertion is constrained by the number of V gene segments, the light chain locus may have one, or more or all of the light dog V gene segments. It may comprise at least 50% of the dog light chain variable (V) genes for kappa and/or lambda, such as at least 60%, at least 70%, at least 80%, at least 90%, and in one aspect all of the dog kappa and/or lambda V genes. In one embodiment the rodent or rodent cell genome may comprise at least 5, 10, 20, 30, 40, 50, 60, 70, 80, 90, 100, 110, 120, 130, 140, 150, or at least 160 dog IGL lambda V region genes. In one preferred embodiment the rodent genome comprises at least 160 dog light chain lambda V region genes. In one embodiment the rodent or rodent cell genome may comprise at least 10, 15, 16, 17, 18 or 19 dog IGL kappa V region genes. In one preferred embodiment the rodent genome comprises at least 19 dog light chain kappa V region genes.

For example, where only the light chain insertion is constrained, the heavy chain locus may have one, or more or all of the heavy chain dog IGH V gene segments. It may comprise at least 50% of the dog heavy chain variable (V) genes, such as at least 60%, at least 70%, at least 80%, at least 90%, and in one aspect all of the dog heavy chain V genes. In this aspect the rodent or rodent cell genome may comprise at least 4, 5, 10, 15 or 20, dog IGH V region genes, such as at least 30, 40, 50, 60 70, or at least 80 dog V region genes. In one preferred aspect the rodent genome comprises at least 83 dog IG heavy chain V region genes.

References herein to genes may, where appropriate, be references to gene segments. Use of the term ‘gene’ is not intended to exclude such features being equivalently disclosed in relation to a gene segment, unless where otherwise dictated by context or necessity.

Similarly, references herein to gene segments may, where appropriate, be references to genes. Use of the term ‘gene segment’ is not intended to exclude such features being equivalently disclosed in relation to a gene, unless where otherwise dictated by context or necessity.

The preferred dog DNA is beagle DNA, and therefore preferred cells and rodents comprise beagle DNA gene segments, as disclosed herein. The use of beagle regulatory sequences is also preferred. The beagle DNA may be provided as genomic DNA.

In all embodiments and aspects of the invention the dog gene segments may be located in the rodent genome upstream of a rodent constant region, suitably upstream of the heavy chain constant region for inserted dog heavy chain variable region gene segments and suitably upstream of a light chain constant region for inserted dog light chain variable region gene segments, such that the rodent or rodent cell is able to produce a chimeric antibody heavy chain, or chimeric light chain, or both, resulting from expression of the inserted variable region gene segments and a host constant region.

Any reference to the location of the variable region upstream of a constant region, such as the rodent constant region, means that there is a suitable relative location of the two genomic portions, encoding the variable and constant regions of the antibody, to allow a chimeric antibody chain to be expressed in vivo in the rodent. In this way the inserted dog DNA and constant region are in functional arrangement with one another for antibody or antibody chain production.

Information concerning, or the nucleic acid comprising, the variable region of a chimeric antibody chain may be obtained from the cells expressing chimeric antibodies using standard techniques. These sequences can be used to generate fully dog antibodies by expression of the nucleic acid encoding the antibody variable region with a dog constant region to generate a dog antibody, for therapeutic use in dogs for example.

The dog DNA may be inserted at the rodent wild-type constant region located at the wild type locus, suitably between the rodent constant region and the host VDJ or VJ region. The rodent constant region expressed with the dog variable region is preferably the rodent wild-type constant region located at the wild type locus, as appropriate for the dog heavy or light chain VDJ or VJ. In one aspect the IGH variable region genes are inserted downstream of the heavy chain J region, and upstream of the Emu enhancer.

In one aspect the IGH variable region genes are inserted downstream of the mouse heavy chain J region, and upstream of the Emu enhancer. In one aspect the rodent is a mouse and insertion of IGH V region genes is made at position 114666435 of the mouse genome, on mouse chromosome 12. In one aspect the insertion of IGL lambda V region genes is made at position 19047551 of the mouse genome, on chromosome 16. In one aspect the insertion of IGL kappa V region gene or genes is made at position 70674755 of the mouse genome, on chromosome 6.

Preferably the inserted dog V(D)J gene segments can undergo V(D)J rearrangement to form an antibody chain in the rodent. However, where a single rearranged heavy or light chain variable region is inserted, or a complete antibody chain VDJC or VJC is inserted, no gene segment rearrangement may be needed.

References herein specifically to ‘mouse’ may, where appropriate, be equivalently references to ‘rodent’. Use of the term ‘mouse’ is not intended to exclude such features being equivalently disclosed in relation to a rodent, unless where otherwise dictated by context or necessity. Preferred embodiments of the invention are directed to a mouse (or a mouse cell).

In an alternative, the inserted dog gene segments are located (inserted) into the genome in functional arrangement with a dog constant region, such that the rodent is able to produce an antibody chain resulting from the expression of the inserted dog VDJ gene segments with a dog constant region, and/or an antibody chain resulting from the expression of the inserted dog VJ gene segments with a dog constant region.

One possibility is the expression of a fully dog antibody light chain with a chimeric heavy chain having a dog VDJ and mouse heavy chain constant region. One possibility is the expression of a fully dog antibody heavy chain with a chimeric light chain having a dog VJ and rodent heavy chain constant region.

References herein such as ‘the inserted dog gene segments’ and ‘the inserted dog DNA’ may refer to the dog sequences which are present in the genome of the rodent or rodent cell, and therefore have been inserted in the genome of the rodent or rodent cell.

In one aspect the inserted dog DNA comprises at least 50% of the dog heavy chain diversity (D) genes, such as at least 60%, at least 70%, at least 80%, at least 90%, and in one aspect all of the dog D genes.

In one aspect the inserted dog DNA comprises at least 50% of the dog heavy chain joining (J) genes, such as at least 60%, at least 70%, at least 80%, at least 90%, and in one aspect all of the dog J genes.

In one aspect the inserted dog DNA comprises at least 50% of the dog light chain joining (J) genes, such as at least 60%, at least 70%, at least 80%, at least 90%, and in one aspect all of the dog light chain J genes.

The rodent or rodent cell genome may comprise at least 1, 2, 3, 4, 5 or 6 IGHD region gene segments from a dog. The rodent or rodent cell genome may comprise at least 1, 2, 3, 4, 5 or 6 IGHJ region genes from a dog. The rodent or rodent cell genome may comprise at least 1, 2, 3, 4, or 5 IGL kappa J region genes from a dog. The rodent or rodent cell genome may comprise at least 1, 2, 3, 4, 5, 6, 7, 8, or 9 IGL lambda J region genes from a dog.

The number of dog genes referred to above in any aspect may also be further increased, and in one aspect is doubled, in the case of a homozygote having an insertion at both alleles.

One possible insertion of dog V gene segments as described above is upstream of the mouse constant region to generate a chimeric antibody chain expressed at the endogenous locus.

In other aspects the inserted dog DNA is located in the rodent genome at a site which is distinct from that of the naturally occurring heavy or light constant region, such as on a different chromosome. The insertion may be at a random location into the rodent genome. In this case the insertion of the VDJ or VJ region genes is accompanied by a constant region, and preferably also by a 3′ enhancer, from the rodent or from the dog. One preferred embodiment is the use of a rodent constant region and rodent 3′ enhancer with dog VDJ or VJ regions, or the use of a dog constant region and dog 3′ enhancer with dog VDJ or VJ regions. In one aspect the dog gene segments(s) are located in the genome in functional arrangement with the constant region such that the rodent is able to produce an antibody chain.

The invention also specifically contemplates cells and rodents having an insertion of dog genes in association with a dog constant region (encoding a “fully” dog antibody chain) at an endogenous rodent IG locus, such as DNA encoding a dog lambda V and J and C genes.

In one aspect the fully dog VJC lambda antibody chain described above is inserted into mouse lambda locus, preferably between last rodent C gene and 3′ enhancer.

In one aspect the rodent or rodent cell comprises one or more dog IGL lambda V region genes, one or more dog IGL lambda J region genes and one or more dog lambda constant regions located at the rodent cell kappa locus, for example within the locus or upstream or downstream of the rodent kappa constant region. Preferably the insertion is upstream of the IGL kappa locus constant region, such that the IGL lambda V and J region genes are expressed with the IGL kappa constant region. Suitably the insertion locates the dog genes at approximately the same position as the native rodent kappa genes, potentially deleting or displacing them, for example there being the same or substantially the same distance from the last inserted 3′ lambda J gene to the rodent kappa constant region gene as there is from the last rodent kappa 3′ J gene to the kappa constant region. In one aspect the insertion is within 1, 2, 3, 4, 5, 6, 7, 8, 9, or 10 kb of the boundary (upstream or downstream) of the rodent immunoglobulin kappa locus. The mouse kappa light chain is naturally expressed at a higher level than the mouse lambda light chain, and insertion of the dog DNA at this kappa locus can provide high levels of expression of the dog lambda chain V region genes.

In one aspect the dog lambda V and/or J gene segments are associated with dog regulatory or scaffold sequences.

In another aspect the dog lambda V and/or J gene segments are associated with regulatory or scaffold sequences from a rodent, such as the regulatory or scaffold sequences native to the rodent cell used for insertion of the dog DNA. In one aspect where dog lambda V genes are inserted at the rodent kappa locus, the rodent kappa locus, or part thereof, may be deleted or rendered non-functional, for example by mutation. Inactivation or rendering an endogenous mouse or rodent K light chain locus nonfunctional to increase expression of lambda light chain is described, for example, in WO2021003149.

References herein to gene segments being associated with regulatory or scaffold sequences, and similar such statements, may be references that the gene segments are operably linked to the regulatory or scaffold sequences. The term ‘associated’ in this context means ‘operably linked’ and may be interchangeable therewith.

In one aspect the rodent genome is homozygous for the inserted dog genes. Such homozygous rodents are preferred for use in the generation of dog antibodies by immunization. The insertion may be at the native rodent Ig loci. In another aspect the rodent genome may be heterozygous for the insertion of dog genes at one, or 2, or three loci, such as immunoglobulin loci.

The rodent genome may be heterozygous for the insertion of dog genes at the kappa locus.

Where the inserted dog DNA at the constrained IgH locus comprises at least 4 IGH variable region genes, preferably one of those is V3-38.

In one aspect the rodent genome is modified to reduce or prevent expression of fully rodent antibodies that have both variable and constant regions from the rodent. This may be by inversion of all or part of the rodent VDJ region and/or VJ region, or by a deletion of all or part of the rodent VDJ region and/or VJ region, or by an insertion into the endogenous rodent VDJ and/or VJ region of the genome. For example, an insertion of dog DNA at an Ig locus upstream of the rodent constant region will move the host rodent V(D) and J gene segments further away from the rodent constant region and reduce expression or inactivate the host rodent antibody expression from that locus. In one aspect the rodent VDJ or VJ region or a part thereof is deleted.

In one aspect all or a number of the rodent V region genes are deleted, such as:
at least 50%, preferably at least 75% or at least 90%, or all, of the rodent IGH V gene segments and/or D gene segments and/or J gene segments, and/or
at least 50% preferably at least 75% or at least 90%, or all, of the rodent IGL V gene segments and/or J gene segments from kappa and/or lambda.

In one aspect the rodent is a mouse, and the genome comprises a deletion of one or some or all of the mouse IGH V region genes, preferably from V1-85 to V5-2. In one aspect the rodent is a mouse, and the genome comprises a deletion of one or some or all of the mouse IGL kappa V region genes, preferably from V3-1 to V2-137. In one aspect the rodent is a mouse, and the mouse heavy chain D and J region genes are retained in the genome upstream of the inserted dog heavy chain variable region genes. In one aspect the rodent IGL lambda genes are not deleted from the rodent genome.

In one aspect one or both alleles of the rodent kappa locus are deleted or inactivated, in whole or in part, by insertion of dog DNA at the rodent kappa locus.

In one aspect the rodent kappa locus is inactivated wholly or partially, for example, by insertion, or by deletion or by inversion.

In one aspect the rodent lambda locus is inactivated, wholly or partially, for example, by insertion, or by deletion or by inversion.

In one aspect the rodent heavy chain locus is inactivated, wholly or partially, for example, by insertion, or by deletion or by inversion.

The dog variable region gene segments(s) are suitably inserted upstream of rodent constant region, the latter comprising all of the DNA required to encode the full constant region or a sufficient portion of the constant region to allow the formation of an effective chimeric antibody capable of specifically recognising an antigen. Reference to a chimeric antibody or antibody chain having a rodent constant region herein therefore is not limited an antibody chain having the complete constant region but also includes chimeric antibodies or chains which have a part of the constant region, sufficient to provide one or more effector functions seen in antibodies occurring naturally in the rodent. Effector functions include the ability to interact with Fc receptors, and/or bind to complement. Likewise, the dog variable region DNA may be located in the host genome such that it forms a chimeric antibody chain with all or part of a rodent constant region to form an antibody chain or a part thereof respectively.

Preferably the rodent genome comprises all the dog lambda constant region DNA and intervening regions.

In one aspect the inserted dog DNA is capable of being expressed with different rodent constant regions through isotype switching.

In one aspect the inserted dog DNA is capable of being expressed with a different rodent constant region through trans-switching.

In one embodiment one, or more, or all of the inserted dog V, D or J region gene segments is associated with a dog regulatory sequence, preferably from the same dog breed.

In one aspect at least one rodent enhancer or other control sequence, such as a switch region, is maintained in functional arrangement with the rodent constant region. In this way the effect of the enhancer or other control sequence may be exerted in whole or in part in the cell or transgenic rodent.

In one aspect one or more rodent control sequences, such as the Emu enhancer sequence, is maintained upstream of the rodent Mu constant region, suitably in its native position with respect to the distance from the constant region.

In one aspect one or more rodent control sequences such as an enhancer sequence(s) is/are maintained downstream of the rodent constant region, suitably in its native position with respect to the distance from the constant region.

In one aspect the rodent Smu switch sequence is maintained upstream of the rodent Mu constant region, suitably in its native position with respect to distance from the constant region.

In such location the rodent enhancer or switch sequence is suitably operative in vivo with the host constant region sequence(s).

In a further aspect one or more of the promoter elements, or other control elements, of the dog V, D or J region genes is/are optimised in the genome to interact with the transcriptional machinery of the rodent.

In one aspect the rodent or rodent cell genome comprises one or more dog promoters, or enhancers, and/or other control elements associated with the dog V, D or J gene segments. In one aspect the one or more dog control regions, such as promoters or enhancers or switch regions, replace one or more rodent promoters or enhancers or switch regions respectively. The dog control sequences are suitably maintained in functional arrangement with a constant region such that the effect of the control sequence(s) may be exerted in whole or in part in the cell or transgenic rodent.

In one aspect at least one or more of the inserted dog V, D or J gene segments is associated with a regulatory sequence, such as a recombination signal sequence (RSS), from the same dog, optionally wherein the regulatory sequences direct the successful recombination of the V, D or J gene segment or segments.

In one aspect, the ‘same’ dog refers to the same breed of dog. In one aspect, the same dog may be precisely the same animal.

In one aspect, at least one or more of the inserted dog V, D or J gene segments is directly associated cis or trans with a regulatory sequence or flanked on one or both sides with a regulatory sequence, optionally wherein the one or more gene segments is directly flanked by the regulatory sequence.

In one aspect the regulatory sequences comprise a promoter preceding an individual V gene segment, and/or a splice site within an individual V gene segment, and/or a recombination signal sequences for V(D)J recombination downstream of a V gene segment, flanking a D gene segment or upstream of a J gene segment.

In one aspect a V, D or J sequence of the inserted dog is flanked by an RSS sequence from the same dog. For example, a dog RSS sequence may be used with dog V, D and/or J sequences. It will be appreciated that this can be provided by insertion of a genomic fragment from the dog into the rodent genome. In a further aspect, the invention provides a method of replacing, in whole or part, in a rodent cell an endogenous immunoglobulin variable region gene locus with a dog gene locus comprising: obtaining a cloned genomic fragment or a synthetic sequence containing, in whole or in part, the companion gene locus comprising at least one V, or D (for the heavy chain) or J gene segments, and at least one associated regulatory sequence, and insertion of the dog DNA into the genome of a rodent, suitably at an endogenous mouse immunoglobulin locus, preferably the heavy or light chain rodent locus corresponding to the nature of the inserted dog DNA.

In one aspect the inserted dog DNA is associated with rodent regulatory sequences which allow for V(D)J recombination in the rodent. Such an approach is disclosed, for example, in US20170306352.

In one aspect the regulatory sequences are non-coding regulatory sequences which comprise the following sequences of endogenous host origin: promoters preceding each V gene segment coding sequence, introns, splice sites, and recombination signal sequences for V(D)J recombination. In another aspect the regulatory sequence is one or more of: promoters preceding each V gene segment coding sequence, introns, splice sites, and recombination signal sequences for V(D)J recombination, all of which are of endogenous host origin.

In another aspect a partly canine immunoglobulin locus generated in the invention comprises one or more of the following sequences of endogenous host origin: ADAM6A or ADAM6B gene, a Pax-5-Activated Intergenic Repeat (PAIR) elements, or CTCF binding sites from a heavy chain intergenic control region 1.

In one aspect the invention relates to a transgenic mouse with a genome in which an entire endogenous immunoglobulin variable gene locus has been deleted and replaced with an engineered partly canine immunoglobulin locus comprising dog immunoglobulin variable gene V H, D and J H and/or dog V L and J L coding sequences as described herein, and mouse immunoglobulin variable gene locus non-coding regulatory sequences, wherein the engineered partly canine immunoglobulin locus of the transgenic mouse is functional and expresses immunoglobulin chains comprised of canine variable domains and mouse constant domains. The mouse may be a mouse as described in US20170306352, the disclosure of which is incorporated by reference. The mouse may be a mouse as described in WO2021003149 the disclosure of which is incorporated by reference.

In one aspect the inserted dog DNA at the constrained locus is not a genomic DNA fragment. In order to insert the gene segment or segments with the highest natural representation, the inserted DNA may comprise DNA in which the gene segments are not in their natural germline configuration and in genomic DNA. The inserted dog DNA may be a minigene having multiple different V gene segments, for example.

Inserted dog V gene segments at the constrained locus may be arranged with gene segment or gene segments of most interest located closest to the constant region. For example, the IGHV 3-38 gene segment may be the most closely located to the constant region to maximize expression. Gene segments located closer to the constant region are usually expressed at higher levels. Alternatively, the gene segments with the highest natural occurrence in the dog antibody population may be located further from the constant region, to provide a balanced repertoire.

The present invention provides a rodent or rodent cell which expresses a limited number of preferred V gene segments, such as one gene segment, and includes as a preferred feature the presence of only a single V, D and J gene segment for the heavy chain variable region, and/or a single V and J for the light chain variable region in the genome. In this way the rodent will generate only a single type of variable region for the heavy chain, and/or a single type of variable region for the light chain of the dog antibody chain that is produced.

In an aspect, the term ‘single type of variable region’ refers to a variable region with only one V, (one D,) and one J gene segment sequence (element). Such an arrangement can provide a common light chain, useful in the generation of bispecific antibodies, where a single light chain is desired, and is expressed with 2 different heavy chains in the bispecific antibody.

Therefore the invention also relates to a method for the generation of a bispecific antibody, the method comprising immunizing a rodent as disclosed herein with an antigen, the rodent comprising only a single type of dog light chain variable region (single dog V and J gene segments, dog VJ) in the rodent genome, and selecting for a bispecific antibodies capable of binding to that antigen, and suitably also to a second preferred antigen target.

The invention also relates to a bispecific antibody obtained or obtainable from a method of the invention, wherein the antibody light chain is obtained from expression of a preferred V gene segment as disclosed herein, suitably from a rodent of the invention. Suitably the bispecific has a preferred light and heavy chain pairing as disclosed herein.

Any bispecific antibody format may be used, such as any of those disclosed in Brinkmann U and Kontermann R E, MAbs. 2017 February-March; 9(2): 182-212.

The dog V gene segment chosen to be used in the common light chain may be based on frequency (Table 1b and Table 1c), number of different heavy chain dog V gene segment pairings (Table 3), or a combination of both. In an aspect, the dog V gene segment chosen to be used in the common light chain is selected from the list comprising the top 20, 10, 5, 4, 3, 2, or 1 dog V gene segment(s) of Table 1b or Table 1c. In an aspect, the dog V gene segment chosen to be used in the common light chain is selected from the list comprising the top 20, 10, 5, 4, 3, 2, or 1 dog V gene segment(s) of Table 3. In an aspect, the dog V gene segment chosen to be used in the common light chain is present in the top 20, 10, 5, 4, 3, 2, or 1 dog V gene segment(s) of both Table 1b and Table 3. In an aspect, the dog V gene segment chosen to be used in the common light chain is present in the top 20, 10, 5, 4, 3, 2, or 1 dog V gene segment(s) of both Table 1c and Table 3. The term ‘top’ used with reference to Table 1b and Table 1c means the most frequently used dog V gene segment(s) in these tables. The term ‘top’ used with reference to Table 3 means the dog V gene segment(s) in this table with the highest number of different heavy chain dog V gene segment pairings.

The term “bispecific antibody” means an antibody which comprises specificity for two target molecules and includes formats such as DVD-Ig (see DiGiammarino et al., “Design and generation of DVD-Ig™ molecules for dual-specific targeting”, Meth. Mo. Biol., 2012, 889, 145 156), mAb2 (see WO2008/003103), FIT-Ig (see WO2015/103072), mAb-dAb, dock and lock, Fab-arm exchange, SEEDbody, Triomab, LUZ-Y, Fcab, KA-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. For a review of bispecific formats, see Spiess, C., et al., Mol. Immunol. (2015). In another embodiment, the bispecific molecule comprises an antibody which is fused to another non-Ig format, for example a T-cell receptor binding domain; an immunoglobulin superfamily domain; an agnathan variable lymphocyte receptor; a fibronectin domain (e.g., an Adnectin™); an antibody constant domain (e.g., a CH3 domain, e.g., a CH2 and/or CH3 of an Fcab™) wherein the constant domain is not a functional CH1 domain; an scFv; an (scFv)2; an sc-diabody; an scFab; a centyrin and an epitope binding domain derived from a scaffold selected from CTLA-4 (Evibody™); a lipocalin domain; Protein A such as Z-domain of Protein A (e.g., an Affibody™ or SpA); an A-domain (e.g., an Avimer™ or Maxibody™); a heat shock protein (such as and epitope binding domain derived from GroEI and GroES); a transferrin domain (e.g., a trans-body); ankyrin repeat protein (e.g., a DARPin™); peptide aptamer; C-type lectin domain (e.g., Tetranectin™); human γ-crystallin or human ubiquitin (an affilin); a PDZ domain; scorpion toxin; and a kunitz type domain of a human protease inhibitor.

Antibody structure has been exploited to engineer a variety of different antibody formats to target disease in humans. An example of such an engineered antibody format is bispecific antibodies. Bispecific antibodies bind to two different targets and are therefore capable of simultaneously binding to two different epitopes. One area of interest is T cell directed bispecific antibodies for efficient tumour killing. Bispecific antibodies can have “two-target” functionality and bind to two different surface receptor or ligands thus influencing multiple disease pathways. Bispecific antibodies can also place two targets in close proximity, either to support protein complex formation on one cell or to trigger contact between cells. Bispecific antibodies formats vary in many ways including their molecular weight, number of antigen-binding sites, spatial relationship between different binding sites, valency for each antigen, ability to support secondary immune functions and pharmacokinetic half-life. These diverse formats provide great opportunity to tailor the design of bispecific antibodies to match the proposed mechanisms of action and the intended clinical application (Kontermann and Brinkmann Bispecific Antibodies Drug Discovery Today Volume 20, Number 7, 2015).

Production of bispecific antibodies of the IgG type by co-expression of the two light and two heavy chains in a single host cell can be highly challenging because of the low yield of desired bispecific IgGs and the difficulty in removing closely related mis-paired IgG contaminants. This reflects that heavy chains form homodimers as well as the desired heterodimers—the so-called heavy chain-pairing problem. Additionally, light chains can mis-pair with non-cognate heavy chains—the so-called light chain pairing problem. Consequently, co-expression of two antibodies can give rise to up to nine unwanted IgG species in addition to the desired bispecific antibody.

Various approaches are described in the art in order to promote heterodimerisation, i.e. the formation of a certain bispecific antibody of interest for human therapy, thereby reducing the content of undesired homodimers in the resulting mixture.

In one embodiment, the antibody is a multispecific antibody or fragment thereof. A multispecific protein, e.g. a multispecific antibody, binds to at least two different targets, i.e. is at least bispecific. Thus, in one embodiment, the antibody is a bispecific antibody or fragment thereof. In other embodiment, the multispecific antibody or fragment thereof binds to three, four or more targets.

A bispecific antibody has specificity for no more than two epitopes. A bispecific antibody is characterized by a first immunoglobulin variable domain sequence which has binding specificity for a first epitope and a second immunoglobulin variable domain sequence that has binding specificity for a second epitope. In one embodiment, the first and second epitopes are on the same antigen, e.g., the same protein (or subunit of a multimeric protein).

In one embodiment, the first and second epitopes overlap. In an embodiment, the first and second epitopes do not overlap. In one embodiment, the first and second epitopes are on different antigens, e.g., the different proteins (or different subunits of a multimeric protein). In another embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In a further embodiment, a bispecific antibody molecule comprises an antibody having binding specificity for a first epitope and an antibody having binding specificity for a second epitope. In one embodiment, a bispecific antibody molecule comprises an antibody, or fragment thereof, having binding specificity for a first epitope and a half antibody, or fragment thereof, having binding specificity for a second epitope. In one embodiment, a bispecific antibody or fragment thereof comprises a Fab having binding specificity for a first epitope and a Fab having binding specificity for a second epitope.

Bispecific antibodies of the invention based on the IgG format, comprising of two heavy and two light chains can be produced by a variety of methods known in the art. For instance, bispecific antibodies may be produced by fusing two antibody-secreting cell lines to create a new cell line or by expressing two antibodies in a single cell using recombinant DNA technology. These approaches yield multiple antibody species as the respective heavy chains from each antibody may form monospecific dimers (also called homodimers), which contain two identical paired heavy chains with the same specificity, and bispecific dimers (also called heterodimers) which contain two different paired heavy chains with different specificity. In addition, light chains and heavy chains from each antibody may randomly pair to form inappropriate, non-functional combinations. This problem, known as heavy and light chain miss-pairings, can be solved by choosing antibodies that share a common light chain for expression as bispecifics. Methods to address the light chain-heavy chain mispairing problem include the generation of bispecific antibodies using a single light chain. This requires heavy-light chain engineering or novel antibody libraries that utilize a single light chain that limits the diversity. In addition, antibodies with a common light chain have been identified from transgenic mice with a single light chain. Another approach is to swap the CH1 domain of one heavy chain with CL domain of its cognate light chain (Crossmab technology). Also covered are scFv formats.

Methods for making bispecific antibodies are described in WO2019008123 and WO2014160179, which are incorporated by reference.

The invention also relates to a method for the generation of a bispecific antibody, the method comprising immunizing a first rodent as disclosed herein with a first antigen, the rodent comprising only a single dog lambda light chain variable region (single dog V and J gene segments, dog VJ) in the rodent genome, and selecting for an antibody capable of binding to said first antigen, and immunizing a second rodent as disclosed herein with a second antigen, the rodent comprising the same single dog lambda light chain variable region (single dog V and J gene segments, dog VJ) in the rodent genome, and selecting for an antibody capable of binding to said second antigen, optionally wherein the single dog lambda light chain V gene segment is selected from the list comprising dog lambda V1-138, V1-136, V1-141, V1-48, V1-55, V1-103, V1-41, V8-153, V1-75, V1-147, V8-128, V1-58, V1-100, V1-125, V1-84, V1-46, V2-8, V3-21, and V1-149.

In one aspect, the single lambda dog light chain V gene segment is V1-138. In one aspect, the single lambda dog light chain V gene segment is V3-3.

The invention also relates to a bispecific antibody obtained or obtainable from a method of the invention, wherein the dog antibody light chain is obtained from expression of a single preferred lambda V gene segment as disclosed herein, suitably from a rodent of the invention. Suitably the bispecific has a preferred light and heavy chain pairing as disclosed herein. In one aspect, the single lambda dog V gene segment is selected from the list comprising dog lambda V1-138, V1-136, V1-141, V1-48, V1-55, V1-103, V1-41, V8-153, V1-75, V1-147, V8-128, V1-58, V1-100, V1-125, V1-84, V1-46, V2-8, V3-21, and V1-149.

The present invention provides a rodent or rodent cell which includes a single dog lambda V and J for the light chain variable region in the genome. In this way the rodent will generate only a single type of variable region for the light chain of the dog antibody chain that is produced. In one aspect, the single dog lambda light chain V gene segment is V1-138. In one aspect, the single lambda dog light chain V gene segment is V3-3.

The invention also relates to a method for the generation of a bispecific antibody, the method comprising immunizing a first rodent as disclosed herein with a first antigen, the rodent comprising only a single dog kappa light chain variable region (single dog V and J gene segments, dog VJ) in the rodent genome, and selecting for an antibody capable of binding to said first antigen, and immunizing a second rodent as disclosed herein with a second antigen, the rodent comprising the same single dog kappa light chain variable region (single dog V and J gene segments, dog VJ) in the rodent genome, and selecting for an antibody capable of binding to said second antigen, optionally wherein the single dog kappa light chain V gene segment is selected from the list comprising dog kappa V2-8, V2-7, V2-4, V2-5, and V2-11.

In one aspect, the single kappa dog light chain V gene segment is V2-8. In one aspect, the single kappa dog light chain V gene segment is V2-7.

The invention also relates to a bispecific antibody obtained or obtainable from a method of the invention, wherein the dog antibody light chain is obtained from expression of a single preferred kappa V gene segment as disclosed herein, suitably from a rodent of the invention. Suitably the bispecific has a preferred light and heavy chain pairing as disclosed herein. In one aspect, the single kappa dog V gene segment is selected from the list comprising dog kappa V2-8, V2-7, V2-4, V2-5, and V2-11.

The present invention provides a rodent or rodent cell which includes a single dog kappa V and J for the light chain variable region in the genome. In this way the rodent will generate only a single type of variable region for the light chain of the dog antibody chain that is produced. In one aspect, the single dog kappa light chain V gene segment is V2-8. In one aspect, the single lambda dog light chain V gene segment is V2-7.

The invention also relates to a specific antibody wherein both light chains are identical and are selected from the list comprising dog lambda V1-138, V1-136, V1-141, V1-48, V1-55, V1-103, V1-41, V8-153, V1-75, V1-147, V8-128, V1-58, V1-100, V1-125, V1-84, V1-46, V2-8, V3-21, and V1-149.

In an embodiment, both light chains are dog lambda V1-138. In an embodiment, both light chains are dog lambda V3-3. In an embodiment, both light chains are dog kappa V2-8.

In one aspect the rodent cell of the invention is a rodent ES cell, rodent hematopoietic stem cell or other cell capable of developing into a rodent able to produce a repertoire of antibody chains comprising a variable region encoded by dog DNA, such as chimeric antibody heavy chains or chimeric antibody light chains, or fully dog antibody chains or antibodies encoded by dog variable regions having variable and constant regions.

In one aspect the cell of the invention is a rodent ES cell or an induced pluripotent stem cell (iPS cell). Such cells are suitable for the insertion of dog DNA to generate rodents expressing antibody chains as described herein. In one aspect the cell is an isolated rodent cell. In one aspect the cell is an isolated rodent B cell.

The rodent or rodent cell of the invention is preferably a mouse or rat, or mouse or rat cell (such as a mouse or rat ES cell) and is preferably a mouse or mouse ES cell. The ES cell may be of mouse cell strain 129 or C57BL, such as strain C57BL/6N, C57BL/6J, 129S5 or 129Sv strains, or in a cell which has a hybrid genome which comprises 129 and/or C57BL genomic DNA.

The invention also relates to a cell line which is grown from or otherwise derived from cells as described herein, including an immortalized cell line.

The cell or cell line of the invention may comprise dog V, (D) or J genes in unrearranged configuration or after rearrangement following in vivo maturation.

The invention also relates to a cell or cell line, such as a CHO cell line, expressing an antibody chain having a variable region which is obtainable by immunizing a rodent of the invention with an antigen, or where the nucleic acid sequence of the variable region may be identified from, or has been identified from a rodent or rodent cell as described herein, or from a repertoire of antibodies described herein. The antibody chain may be a chimeric antibody heavy chain or is preferably a fully dog antibody chain. The cell or cell line expressing the antibody chain or antibody may be a CHO cell, or other mammalian cell line suitable for the production of a therapeutic for animal use. The cell may be immortalized by fusion to a tumor cell to provide an antibody producing cell and cell line or be made by direct cellular immortalization.

The present invention also relates to vectors for use in the invention. In one aspect such vectors are bacterial artificial chromosomes (BACs) comprising all or a part of the dog IG locus suitable for insertion into an ES cell. It will be appreciated that other cloning vectors may be used in the invention, and therefore reference to BACs herein may be taken to refer generally to any suitable vector. The vector may comprise one or more selectable markers and/or one or more site specific recombination sites. In one aspect the vector comprises 2 or more, such as 3, heterospecific and incompatible site-specific recombination sites. In one aspect the site-specific recombination sites may be loxP sites, or variants thereof, or FRT sites or variants thereof. In one aspect the vector comprises one or more transposon ITR (inverted terminal repeat) sequences.

Suitable BACs containing dog DNA are available as the CHORI-82 BAC library from the BACPAC Resources Center of the Children's Hospital Oakland Research institute.

In one aspect some or all of the inserted DNA is from a boxer dog. Preferably the cell or rodent comprises boxer dog V, D and J gene segments from the CHORI-82 BAC library.

In one aspect one or more dog gene segment alleles used in the present invention are the reference alleles for each dog gene. These are those of CanFam3.1—see assembly accession—GCA_000002285.2, produced September 2011 and last updated May 2016. In one aspect at least 90%, at least 95% and preferably all of the dog gene segments that have been inserted are the dog reference alleles from CanFam 3.1. In one aspect, at least 50, 60, 70, 80, 90 or 100% of the inserted V gene segments are dog V gene reference alleles, and/or at least 50, 60, 70, 80, 90 or 100% of the inserted D gene segments are dog D gene reference alleles, and/or at least 50, 60, 70, 80, 90 or 100% of the inserted J gene segments are dog J gene reference alleles, and combinations of these. Additional dog reference alleles may be available in canFam4 (UU_CFam_GSD_1.0/canFam4, https://www.nature.com/articles/s42003-021-01698-x#MOESM1).

In one aspect, references to dog gene segments (such as IGHV3-38) are references to the equivalent gene segments of CanFam3.1. However, it will be understood that such sequences for use in the invention can be different from the exact sequence of CanFam3.1, such as 99% identical, 98% identical, 97% identical, 96% identical or 95% identical. In one aspect, reference to dog gene segments (such as IGHV3-38) are references to gene segments having the relevant sequence in Table 4.

In one aspect, the rodent or rodent cell comprises one or more reference alleles of the dog gene segments. Preferably the V (D) and J region gene segments that are inserted into the genome are all from the same breed of dog or the same dog.

In one aspect the rodent is able to generate a diversity of at least 1×10⁶ different functional chimeric immunoglobulin sequence combinations.

It is noted that reference herein to ‘dog’, or reference herein to ‘canine’, are intended to be interchangeable, and both terms refer to Canis familiaris/Canis lupus familiaris.

The invention also relates to methods for making rodents comprising inserted dog DNA, methods for making antibodies and antibody chains from those rodents, and methods for making pharmaceutical compositions comprising dog antibody chains or antibodies.

A method for producing a rodent or rodent cell disclosed herein, the method comprising inserting into a rodent cell genome:
(i) no more than 8 dog IGHV gene segments, one or more dog IGH D region gene segments and one or more dog IGH J region gene segments, wherein at least one of the IGH V gene segments is selected from the list comprising V3-38, V3-19, V3-35, V3-5, V4-1, V3-41, V3-26 and V1-30; and/or
(ii) no more than 17 dog lambda V gene segments and one or more dog IGH J region gene segments, wherein at least one of the IGH V gene segments is selected from the list comprising V 1-138, 1-141, 1-55, 1-136, 1-75, 1-48, 8-153, 1-147, 1-125, 1-41, 1-149, 1-58, 1-100, 1-46, 3-21, 1-103, and 1-84; and/or
(iii) no more than 5 dog kappa V gene segments and one or more dog J region gene segments, wherein at least one of the dog kappa gene segments is selected from V 2-8, 2-11, 2-5, 2-4 and 2-7;
and wherein the rodent or rodent cell is capable of expressing the dog variable region gene segment or segments in combination with a constant region to form an antibody chain.

Methods for targeted insertion of exogenous DNA at the endogenous mouse locus are well known in the art, such that inserted V, D and J genes can be expressed with the host constant region. The same principles can be applied to insertion of canine DNA. See Murphy et al, Vol 111 no 14, 5153-5158, doi: 10.1073/pnas.1324022111; MacDonald et al vol. 111 no. 14, 5147-5152, doi: 10.1073/pnas.1323896111; and Lee et al, Nature Biotechnology Volume: 32, Pages:356-363 Year published: 2014 DOI:, doi: 10.1038/nbt.2825. Suitable methods for the construction of a chimeric Ig locus encoding canine VDJ and or VJ gene segments with a constant region are disclosed in WO2018189520, the methods of which are incorporated herein by reference.

Preferably the method relates to inserting both light chain and heavy chain dog VDJ and VJ region genes respectively such that an antibody is produced in which both light and heavy chains have a variable region derived from expression of dog DNA.

In one aspect rodent ES cells carrying one or more chimeric loci are used to make chimaeras in which the host embryos are generated from a RAG-1-deficient background, or other suitable genetic background which prevents the production of mature host B and T lymphocytes. This enables all the B and T cells to be derived from the injected ES cells.

The ES cells of the present invention can be used to generate animals using techniques well known in the art, which may for example comprise injection of the ES cell into a blastocyst followed by implantation of chimeric blastocysts into females to produce offspring, which can be bred to produce heterozygous offspring which are then interbred to produce homozygous recombinants having the required insertion. In one aspect the host blastocysts are Rag-deficient, such as RAG-1-deficient.

The invention relates to a chimeric rodent generated by injection of an ES cell of the invention into a blastocyst followed by implantation of chimeric blastocysts into a rodent female to produce offspring.

In one aspect the rodent or rodent cell is a mouse or mouse cell and the mouse ADAM6a and ADAM6b genes are present in the mouse genome and have not been previously deleted then reinserted from the IGH locus.

In one aspect the rodent ADAM6a and ADAM6b genes are located at a position 5′ of the inserted one or more dog V, D and J genes.

In one aspect the rodent IGH D and J genes are present in the rodent genome. In one aspect the rodent IGH D and J genes have not been deleted from the rodent genome. In one aspect the rodent IGH D and J genes are located at a position 5′ of the inserted one or more dog V, D and J gene segments.

The invention also relates to a method for producing an antibody or antibody chain specific to a desired antigen, the method comprising immunizing a rodent as disclosed herein with the desired antigen and recovering the antibody or antibody chain, singularly or as part of a complete antibody, or recovering a cell producing the antibody or antibody chain, singularly or as part of a complete antibody (see e.g. Harlow, E. & Lane, D. 1998, 5th edition, Antibodies: A Laboratory Manual, Cold Spring Harbor Lab. Press, Plainview, NY; and Pasqualini and Arap, Proceedings of the National Academy of Sciences (2004) 101:257-259).

Suitably an immunogenic amount of the antigen is delivered. The invention also relates to a method for detecting a target antigen comprising detecting an antibody produced as above with a secondary detection agent which recognises a portion of that antibody.

The invention also relates to a method for producing an antibody chain or antibody specific to a desired antigen the method comprising immunizing a rodent comprising dog gene segments located upstream of a constant region which is not from a dog, as disclosed herein, and then replacing the constant region of the antibody chain or antibody with that of a dog constant region, suitably by engineering of the nucleic acid encoding the antibody. Preferably the constant region is from the same dog breed. Standard cloning techniques are known to replace the non-human mammal constant region with an appropriate dog constant region DNA sequence—see e.g. Sambrook, J and Russell, D. (2001, 3′d edition) Molecular Cloning: A Laboratory Manual (Cold Spring Harbor Lab. Press, Plainview, NY). Alternatively direct nucleic acid synthesis may be used to generate the fully dog sequence using sequence information from the DNA encoding the chimeric antibody.

The invention also relates to a method comprising identifying dog variable regions by single cell sequencing and making a vector to express a full antibody chain with the corresponding dog constant region. The invention also relates to obtaining these dog sequences by PCR and joining them to an appropriate constant region, such as a dog constant region, by a suitable molecular biology technique such as but not limited to bridge-PCR and Gibson cloning. The DNA may also be synthesized for inclusion in an expression vector.

In a yet further aspect, chimeric antibodies or antibody chains generated in the present invention may be manipulated, suitably at the DNA level, to generate molecules with antibody-like properties or structure, such as:
a dog variable region from a heavy or light chain absent a constant region,
a domain antibody,
a dog variable region with any constant region from either heavy or light chain from the same different species or
a dog variable region with a non-naturally occurring constant region; or a dog variable region together with any other fusion partner. The invention relates to all such chimeric antibody derivatives derived from chimeric antibodies identified according to the present invention.

The invention also relates to a method for making an antibody, or part thereof, the method comprising providing:
(i) a nucleic acid encoding an antibody, or a part thereof, obtained or obtainable according to the present invention; or
(ii) sequence information from which a nucleic acid encoding an antibody obtained or obtainable according to the present invention, or part thereof, can be expressed to allow an antibody to be produced; and expressing the antibody chain.

Also disclosed is a method for producing an antibody chain or part thereof, the antibody chain having a dog variable region, the method comprising expressing in a cell a nucleic acid such as DNA encoding the antibody chain, or a part thereof,

wherein the sequence of the nucleic acid encoding the variable region of the antibody chain is obtained from, or

obtainable by immunising a rodent disclosed herein with an antigen so that antibody chains are produced, or

obtained from an antibody of a repertoire as described herein, optionally including the subsequent steps of:
purifying and/or isolating the antibody chain, and
optionally then formulating the antibody into a pharmaceutically acceptable formulation suitable for administration into a dog.

Also disclosed is a method of making a pharmaceutical composition, the method comprising producing an antibody according to a method disclosed herein and further comprising combining the antibody with a pharmaceutically acceptable carrier or other excipient to produce the composition.

The invention further relates to chimeric antibodies or antibody chains expressed from gene segments identified as important in generating canine antibodies, and nucleic acids encoding the same, such as:

A nucleic acid encoding a chimeric antibody chain, the nucleic acid having a variable domain which comprises any one of the dog IGHV gene segments selected from the list comprising V3-38, V3-19, V3-35, V3-5, V4-1, V3-41, V3-26 and V1-30 and a rodent constant region, optionally wherein the dog V gene segment is a sequence that has undergone somatic hypermutation in a rodent.

An antibody chain having a variable region obtained by expression of any one of dog IGH V3-38, V3-19, V3-35, V3-5, V4-1, V3-41, V3-26 and V1-30 in a rodent, in conjunction with a rodent constant region.

A nucleic acid encoding a chimeric antibody chain, the nucleic acid having a variable domain which comprises any one of the dog lambda V gene segments selected from the list comprising lambda V 1-138, 1-141, 1-55, 1-136, 1-75, 1-48, 8-153, 1-147, 1-125, 1-41, 1-149, 1-58, 1-100, 1-46, 3-21, 1-103 and 1-84, and a rodent constant region, optionally wherein the dog V gene segment is a sequence that has undergone somatic hypermutation in a rodent.

An antibody chain having a variable region obtained by expression of any one of dog lambda V 1-138, 1-141, 1-55, 1-136, 1-75, 1-48, 8-153, 1-147, 1-125, 1-41, 1-149, 1-58, 1-100, 1-46, 3-21, 1-103, and 1-84 in a rodent, in conjunction with a rodent constant region.

A nucleic acid encoding a chimeric antibody chain, the nucleic acid having a variable domain which comprises any one of the dog kappa V gene segments selected from the list comprising 2-8, 2-11, 2-5, 2-4 and 2-7 and a rodent constant region, optionally wherein the dog V gene segment is a sequence that has undergone somatic hypermutation in a rodent.

An antibody chain having a variable region obtained by expression of any one of dog kappa V2-8, 2-11, 2-5, 2-4 and 2-7 in a rodent, in conjunction with a rodent constant region.

The antibody, or antibody chain, or nucleic acid may be obtained or be obtainable from a rodent or rodent cell, or be obtained from a repertoire of antibodies as disclosed herein.

The invention also relates to a part of, or a whole, immunoglobulin molecule comprising dog variable domains and rodent constant domains derived from the constrained insertion of a B cell from a rodent, and a hybridoma cell obtainable or obtained from that B cell, and a part of, or whole, immunoglobulin molecule comprising dog variable domains and rodent constant domains obtainable from or obtained from that hybridoma cell.

In another aspect the invention relates to fragments and functional derivatives of said antibodies and chains disclosed herein, also referred to as parts of antibody chains, and use of said antibodies, chains and fragments in medicine, including diagnosis, and in vitro or ex vivo studies. Functional antibody fragments/parts can include a fragment that is capable of specific binding to an antigen. A functional antibody fragment may be, for example, a FAB or a single chain variable fragment (scFv). The fragment may comprise at least the variable region of the antibody. The fragment may comprise at least the CDR regions. Suitably the parts are functional in that they can bind a desired antigen—preferably the same antigen used to immunize the rodent to stimulate antibody production.

The invention also relates to nucleic acid, such as DNA or RNA, encoding said antibody, antibody chains, or parts thereof. In particular the part may be the variable portion of the antibody chain, which is that part encoded by the dog DNA within the rodent.

In one aspect the antibody or fragment comprises any combination exemplified in the Examples and Figures herein, or any derivative thereof which is a fully dog antibody or a fragment thereof, capable of antigen binding.

Antibodies of the invention may be isolated, in one aspect being isolated from the cell or organism in which they are expressed.

The present invention relates to both polyclonal and monoclonal antibodies of chimeric or fully dog antibodies, which may be produced in response to antigen challenge in rodents of the present invention, or derived therefrom, as described herein, and/or which may comprise the V gene segments identified herein as being highly utilized, such as IGH 3-38. Methods for the generation of both monoclonal and polyclonal antibodies are well known in the art.

Also disclosed herein are pharmaceutical compositions comprising said antibodies and antibody chains, or nucleic acid encoding the antibodies or chains.

The invention relates to a pharmaceutical composition comprising an antibody chain having a variable region comprising any one of dog IGH V3-38, V3-19, V3-35, V3-5, V4-1, V3-41, V3-26 and V1-30, in conjunction with a pharmaceutically acceptable excipient or carrier.

A pharmaceutical composition comprising an antibody chain having a variable region comprising any one of lambda V 1-138, 1-141, 1-55, 1-136, 1-75, 1-48, 8-153, 1-147, 1-125, 1-41, 1-149, 1-58, 1-100, 1-46, 3-21, 1-103, and 1-84 in conjunction with a pharmaceutically acceptable excipient or carrier.

A pharmaceutical composition comprising an antibody chain having a variable region comprising any one of dog kappa V2-8, 2-11, 2-5, 2-4 and 2-7, in conjunction with a pharmaceutically acceptable excipient or carrier

A pharmaceutical composition comprising an antibody, the antibody having:

(i) a heavy chain having a variable region obtained by expression of any one of dog IGH V3-38, V3-19, V3-35, V3-5, V4-1, V3-41, V3-26 and V1-30, and either

(ii) a lambda light chain obtained by expression of any one of lambda V 1-138, 1-141, 1-55, 1-136, 1-75, 1-48, 8-153, 1-147, 1-125, 1-41, 1-149, 1-58, 1-100, 1-46, 3-21, 1-103, and 1-84; or a kappa light chain obtained by expression of any one of kappa V2-8, 2-11, 2-5, 2-4 and 2-7 the antibody being formulated in conjunction with a pharmaceutically acceptable excipient or carrier.

Suitable excipients and carriers are well known in the art and include water, surfactants, carbohydrates (e.g., cyclodextrin derivatives) and amino acids.

The invention also relates to a pharmaceutical composition comprising a fully dog antibody packaged within a delivery vehicle, such as an IV bag, or injection device.

The antibody chain may be a chimeric or fully dog antibody chain.

The invention also relates to primers, specifically the primers of Table 2.

The invention also relates to repertoires of antibodies, however made. For example, the repertoire may be derived from a transgenic mouse or alternatively a synthetic antibody repertoire such as a phage display system utilising the same limited set of immunoglobulin V gene segments as disclosed herein for the inserted heavy and/or light chain dog DNA. Suitable methods for the generation of synthetic antibody repertoires are described for example in WO2018234438.

The invention therefore relates to an antibody heavy chain repertoire, the repertoire comprising antibody heavy chains having dog IGHV gene segments from no more than 8 different dog IGH V gene segments, wherein at least one of the IGH V gene segments is selected from the list comprising V3-38, V3-19, V3-35, V3-5, V4-1, V3-41, V3-26 and V1-30, optionally wherein at least 2, at least 3, at least 4, at least 5, at least 6, at least 7, or all 8 IGH V gene segments is selected from the list comprising V3-38, V3-19, V3-35, V3-5, V4-1, V3-41, V3-26 and V1-30.

Optionally the repertoire comprises antibody heavy chains having dog IgHV gene segments expressed from
(i) V3-38, optionally with up to 7 other dog IGH V gene segments
(ii) V3-38 and V3-19, optionally with up to 6 other dog IGH V gene segments
(iii) V3-38, V3-19 and V3-35, optionally with up to 5 other dog IGH V gene segments
(iv) V3-38, V3-19, V3-35, and V3-5, optionally with up to 4 other dog IGH V gene segments
(V) V3-38, V3-19, V3-35, V3-5, V4-1, optionally with up to 3 other dog IGH V gene segments
(VI) V3-38, V3-19, V3-35, V3-5, V4-1, V3-41, optionally with up to 2 other dog IGH V gene segments
(VII) v3-38, V3-19, V3-35, V3-5, V4-1, V3-41, V3-26, optionally with up to 1 other dog IGH V gene segment
(VII) V3-38, V3-19, V3-35, V3-5, V4-1, V3-41, V3-26 and V1-30

In one embodiment the repertoire comprises antibody heavy chains expressed from IGHV gene segments of dog IGHV families 1, 3 and 4.

Also disclosed is an antibody light chain repertoire, the repertoire comprising antibody light chains having dog lambda V gene segments from no more than 17 different lambda V gene segments, wherein at least one of the V gene segments is selected from the list comprising 1-138, 1-141, 1-55, 1-136, 1-75, 1-48, 8-153, 1-147, 1-125, 1-41, 1-149, 1-58, 1-100, 1-46, 3-21, 1-103 and 1-84, optionally wherein at least 2, at least 3, at least 4, at least 5, at least 6, at least 7, at least 8, at least 9, at least 10, at least 11, at least 12, at least 13, at least 14, at least 15, at least 16, or all 17 lambda V gene segments is selected from the list comprising 1-138, 1-141, 1-55, 1-136, 1-75, 1-48, 8-153, 1-147, 1-125, 1-41, 1-149, 1-58, 1-100, 1-46, 3-21, 1-103 and 1-84.

Optionally the repertoire comprises antibody lambda light chains having dog lambda V gene segments including
(i) 1-138, optionally with up to 16 other dog lambda V gene segments
(ii) 1-138, 1-141 optionally with up to 15 other dog lambda V gene segments
(iii) 1-138, 1-141, 1-55, optionally with up to 14 other dog lambda V gene segments
(iv) 1-138, 1-141, 1-55, 1-136, optionally with up to 13 other dog lambda V gene segments
(V) 1-138, 1-141, 1-55, 1-136, 1-75, optionally with up to 12 other dog lambda V gene segments
(VI) 1-138, 1-141, 1-55, 1-136, 1-75, 1-48, optionally with up to 11 other dog lambda V gene segments
(VII) 1-138, 1-141, 1-55, 1-136, 1-75, 1-48, 8-153, optionally with up to 10 other dog lambda V gene segments

In another embodiment the repertoire comprises antibody lambda light chains expressed from lambda V gene segments of dog lambda families 1, 2, 3, 4, 5 and 8.

Also disclosed is an antibody light chain repertoire, the repertoire comprising antibody kappa light chains having dog kappa V gene segments from no more than 5 different kappa V gene segments, wherein at least one of the V gene segments is selected from the list comprising V2-8, 2-11, 2-5, 2-4 and 2-7, optionally wherein at least 2, at least 3, at least 4, or all 5 kappa V gene segments is selected from the list comprising V2-8, 2-11, 2-5, 2-4 and 2-7.

Optionally the repertoire comprises antibody kappa light chains having dog kappa V gene segments including
(i) 2-8, optionally with up to 4 other dog lambda V gene segments;
(ii) 2-8, 2-11 optionally with up to 3 other dog lambda V gene segments;
(iii) 2-8, 2-11, 2-5 optionally with up to 2 other dog lambda V gene segments;
(iv) 2-8, 2-11, 2-5, 2-4 optionally with up to 1 other dog lambda V gene′ segments; or
(V) 2-8, 2-11, 2-5, 2-4 and 2-7.

Optionally the repertoire comprises antibody kappa light chains having dog kappa V gene segments including
(i) 2-8, optionally with up to 4 other dog kappa V gene segments;
(ii) 2-8, 2-11 optionally with up to 3 other dog kappa V gene segments;
(iii) 2-8, 2-11, 2-5 optionally with up to 2 other dog kappa V gene segments;
(iv) 2-8, 2-11, 2-5, 2-4 optionally with up to 1 other dog kappa V gene segments; or
(V) 2-8, 2-11, 2-5, 2-4 and 2-7.

In another embodiment the repertoire comprises antibody kappa light chains expressed from kappa V gene segments of dog kappa V families 2, 3 and 4

In one embodiment, the repertoire comprises any one of those VH and VL regions that are described in WO2018234438, see in particular, SEQ ID NOs: 1-36 as set out on pages 59 to 62 therein.

The repertoire may comprise one of the following germline VH1 regions: Vs618 (SEQ ID No.: 4 therein), Vs624 (SEQ ID No.: 1 therein), Vs628 (SEQ ID No.: 5 therein) and Vs635 (SEQ ID No.: 2 therein) as disclosed in WO2018234438

The repertoire may comprise one of the following germline VL regions: Vs236 (kappa) (SEQ ID No.: 12 therein), Vs321 (lambda) (SEQ ID No.: 14 therein), Vs323 (lambda) (SEQ ID No.: 16 therein), Vs365 (lambda) (SEQ ID No.: 13 therein) and Vs843 (lambda) (SEQ ID No.: 15 therein) as disclosed in WO2018234438.

The antibodies and antibody chains disclosed herein may be used in methods of prevention or treatment of disease in a dog. In particular, fully dog antibodies, which have a dog constant region, may be used.

The invention thus relates to an antibody or antibody or chain, or part thereof, as disclosed herein, such as an antibody obtained or obtainable from a rodent or rodent cell disclosed herein, or from a repertoire disclosed herein, such as a fully dog antibody made using the sequence information from a rodent or rodent cell, for use in treatment or prevention of disease a dog in need thereof.

The invention also relates to a method of treatment of a dog, the method comprising delivery of an antibody or antibody chain or part thereof as disclosed herein, to a dog in need thereof, the antibody being, for example, an antibody obtained or obtainable from a rodent or rodent cell disclosed herein, or from a repertoire as disclosed herein, or a fully dog antibody made using the sequence information from a rodent or rodent cell.

In a further aspect the invention relates to use of a rodent as described herein as a model for the testing of drugs and vaccines. The invention therefore relates to a method for identification or validation of a drug or vaccine, the method comprising delivering the vaccine or drug to a mammal of the invention and monitoring one or more of: the immune response, the safety profile; the effect on disease.

The invention also relates to a kit comprising an antibody or antibody derivative as disclosed herein and either instructions for use of such antibody or a suitable laboratory reagent, such as a buffer, antibody detection reagent or excipient for formulation with the antibody.

Certain preferred gene segments are disclosed herein, and we have provided the relevant sequences:
IGH V gene segments selected from the list comprising V3-38, V3-19, V3-35, V3-5, V4-1, V3-41, V3-26 and V1-30.
Lambda gene segments selected from 1-138, 1-141, 1-55, 1-136, 1-75, 1-48, 8-153, 1-147, 1-125, 1-41, 1-149, 1-58, 1-100, 1-46, 3-21, 1-103 and 1-84.
Kappa gene segments selected from 2-8, 2-11, 2-5, 2-4 and 2-7.

TABLE 4

| V gene | | SEQ |
|---|---|---|
| segment | Sequence | ID NO |
| Heavy chain | | |
| IGH V3-38 | gaggtgcagctggtggagtctgggggagacctggtgaagcctggggggaccttgagactgtcctgt | 1 |
| | gtggcctctggattcacctttagtagctatgacatgagctgggtccgtcagtctccagggaaggggct | |
| | gcagtgggtcgcagttatttggaatgatggaagtagcacatactacgcagacgctgtgaagggccg | |
| | attcaccatctccagagacaacgccaagaacacgctgtatctgcagatgaacagcctgagagccga | |
| | ggacacggccgtgtattactgtgcgaagga | |
| IGH V3-19 | gaggtgcagctggtggagtctgggggagacctggtgaagcctgcggggtccctgagactgtcctgt | 2 |
| | gtggcctctggattcaccttcagtagctacagcatgagctgggtccgccaggctcctgagaaggggc | |
| | tgcagttggtcgcaggtattaacagcggtggaagtagcacatactacacagacgctgtgaagggcc | |
| | gattcaccatctccagagacaacgccaagaacacagtgtatctgcagatgaacagcctgagagccg | |
| | aggacacggccatgtattactgtgcaaagga | |
| IGH V3-35 | gaggtgcagctggtggagtctgggggagacctggtgaagcctgtgggatccctgagactctcctgt | 3 |
| | gtggcctctggattcaccttcagtagctatgacatgaactgggtccgccaggctccagggaaggggc | |
| | tgcagtgggtcgcatacattagcagtggtggaagtagcacatactatgcagatgctgtgaagggccg | |
| | gttcaccatctccagagacaacgccaagaacacgctgtatcttcagatgaacagcctgagagccga | |
| | ggacacggccatgtattactgtgcgggtga | |
| IGH V3-5 | gaggtgcagctggtggagtctgggggagacctggtgaagcctggggggtccctgagactttcctgt | 4 |
| | gtggcctctggattcaccttcagtagctaccacatgagctgggtccgccaggctccagggaagggg | |
| | cttcagtgggtcgcatacattaacagtggtggaagtagcacaagctatgcagacgctgtgaagggcc | |
| | gattcaccatctccagagacaacgccaagaacacgctgtatcttcagatgaacagcctgagagccg | |
| | aggacacggccgtgtattactgtgcgagtga | |
| IGH V4-1 | gaactcacactgcaggagtcagggccaggactggtgaagccctcacagaccctctctctcacctgt | 5 |
| | gttgtgtccggaggctccgtcaccagcagttactactggaactggatccgccagcgccctgggagg | |
| | ggactggaatggatggggtactggacaggtagcacaaactacaacccggcattccagggacgcat | |
| | ctccatcactgctgacacggccaagaaccagttctccctgcagctgagctccatgaccaccgagga | |
| | cacggccgtgtattactgtgcaagaga | |
| IGH V3-41 | gaggtgcagctggtggagtctgggggagacctggtgaagcctggggggtccctgagactctcctgt | 6 |
| | gtagcctctggattcaccttcagtaactacgacatgagctgggtccgccaggctcctgggaaggggc | |
| | tgcagtgggtcgcagctattagctatgatggaagtagcacatactacactgacgctgtgaagggccg | |
| | attcaccatctccagagacaacgccaggaacacagtgtatctgcagatgaacagcctgagagccga | |
| | ggacacggctgtgtattactgtgcgaagga | |
| IGH V3-26 | gaggtgtagctggtggagtctgggggagacctggtgaagcctggggggtccctgagactctcctgt | 7 |
| | gtgggctctggattcaccttcagtagctactggatgagctgggtccgccaggctccagggaagggg | |
| | ctacagtgggttgcagaaattagcggtagtggaagtagcacaaactatgcagacgctgtgaagggc | |
| | cgattcatcatctccagagacaatgccaagaacacgctgtatctgcagatgaacagcctgagagccg | |
| | aggacacggccatgtattactgtgcaaggga | |
| IGH V1-30 | gaggtccagctggtgcagtctggggctgaggtgaagaagccaggggcatctgtgaaggtctcctgc | 8 |
| | aagacatctggatacaccttcattaactactatatgatctgggtacgacaggctccaggagcagggct | |
| | tgattggatgggacagattgatcctgaagatggtgccacaagttatgcacagaagttccagggcaga | |
| | gtcaccctgacagcagacacatccacaagcacagcctacatggagctgagcagtctgagagctgg | |
| | ggacatagctgtgtactactgtgcgaga | |
| LAMBDA V | | |
| IGLV1-138 | cagtctgtgctgactcagccggcctcagtgtctgggtccctgggccagagggtcaccatctcctgca | 9 |
| | ctggaagcagctccaatgttggttatggcaattatgtgggctggtaccagcagctcccaggaacaag | |
| | ccccagaaccctcatctatgatagtagtagccgaccctcgggggtccctgatcgattctctggctcca | |
| | ggtcaggcagcacagcaaccctgaccatctctgggctccaggctgaggatgaagccgattattactg | |
| | ctcatcctatgacagcagtctcagtggtgg | |
| IGLV1-141 | cagtctgtgctgactcagccgacctcagtgtcggggtcccttggccagagggtcaccatctcctgctc | 10 |
| | tggaagcacgaacaacatcggtattgttggtgcgagctggtaccaacagctcccaggaaaggcccc | |
| | taaactcctcgtgtacagtgttggggatcgaccgtcaggggtccctgaccggttttccggctccaact | |
| | ctggcaactcagccaccctgaccatcactgggcttcaggctgaggacgaggctgattattactgcca | |
| | gtcctttgataccacgcttggtgctca | |
| IGLV 1-55 | cagtctgtgctgactcagccggcctcagtgtctgggtccctgggccagaggatcaccatctcctgca | 11 |
| | ctggaagcagctccaacattggaggtaataatgtgggttggtaccagcagctcccaggaagaggcc | |
| | ccagaactgtcatctatagtacaaatagtcgaccctcgggggtgcccgatcgattctctggctccaag | |
| | tctggcagcacagccaccctgaccatctctgggctccaggctgaggatgaggctgattattactgctc | |
| | aacgtgggatgatagtctcagtgctcc | |
| IGLV1-136 | cagtctgtgctgactcagccggcctcagtgtctgggtccctgggccagagggtcaccatctcctgca | 12 |
| | ctggaagcagctccaacatcggtagaggttatgtgggctggtaccagcagctcccaggaacaggcc | |
| | ccagaaccctcatctatgatagtagtagccgaccctcgggggtccctgatcgattctctggctccagg | |
| | tcaggcagcacagcaaccctgaccatctctgggctccaggctgaggacgaggctgattattactgct | |
| | cagcatatgacagcagtctcagtggtgg | |
| IGLV 1-75 | cagtctgtgctgactcagccggcctcagtgactgggtccctgggccagagggtcaccatctcctgca | 13 |
| | ctggaagcagctccaacatcggtggatataatgttggctggttccagcagctcccgggaacaggcc | |
| | ccagaaccgtcatctatagtagtagtaaccgaccctcgggggtcccggatcgattctctggctccag | |
| | gtcaggcagcacagccaccctgaccatctctgggctccaggctgaggacgaggctgagtattactg | |
| | ctcaacatgggacagcagtctcaaagctcc | |
| IGLV 1-48 | cagtctatgctgactcagccagcctcagtgtctgggtccctgggccagaaggtcaccatctcctgcac | 14 |
| | tggaagcagctccaacatcggtggtaattatgtgggctggtaccaacagctcccaggaataggccct | |
| | agaaccgtcatctatggtaataattaccgaccttcaggggtccccgatcgattctctggctccaagtca | |
| | ggcagttcagccaccctgaccatctctgggctccaggctgaggacgaggctgagtattactgctcat | |
| | catgggatgatagtctcagaggtca | |
| IGLV 8-153 | cagactgtggtaacccaggagccatcactctcagtgtctccaggagggacagtcacactcacatgtg | 15 |
| | gcctcagctctgggtcagtctctacaagtaattaccctggctggtaccagcagacccaaggccgggc | |
| | tcctcgcacgattatctacaacacaagcagccgcccctctggggtccctaatcgcttctctggatccat | |
| | ctctggaaacaaagccgccctcaccatcacaggagcccagcccgaggatgaggctgactattactg | |
| | ttccttgtatacgggtagttacactga | |
| IGLV 1-147 | cagtctgtgctgactcagccggcctcagtgtctgggtccctgggccagagggtcaccatctcctgca | 16 |
| | ctggaagcagctccaacatcggtagaggttatgtgggctggtaccagcagctcccaggaacaggcc | |
| | ccagaaccctcatctatgataatagtaaccgaccctcgggggtccctgatcgattctctggctccaagt | |
| | caggcagcacagccaccctgaccatctctgggctccaggctgaggacgaggctgattattactgctc | |
| | aacatacgacagcagtctcagtggtgg | |
| IGLV 1-125 | cagtctgtgctgactcagccggcctcagtgtccgggtccctgggccagagggtcaccatctcctgca | 17 |
| | ctggaagcagctccaacatcggtagaggttatgtgggctggtaccaacagctcccgggaacaggcc | |
| | ccagaaccctcatctatggtaatagtaaccgaccctcaggggtccccgatcggttctctggctccagg | |
| | tcaggcagcacagccaccctgaccatctctgggctccaggctgaggatgaggctgattattactgct | |
| | catcgtgggacagcagtctcagtgctct | |
| IGLV 1-41 | cagtctgtgctgactcagccagcctccgtgtctgggtccctgggccagagggtcaccatttcctgcac | 18 |
| | tggaagcagctccaacgttggttatagcagtagtgtgggctggtaccagcagttcccaggaacaggc | |
| | cccagaaccatcatctattatgatagtagccgaccctcgggggtccccgatcgattctctggctccaa | |
| | gtctggcagcacagccaccctgaccatctctgggctccaggctgaggatgaggctgattattactgct | |
| | catcttgggacaacagtctcaaagctcc | |
| IGLV 1-149 | cagtctgtgctgactcagccggcctcagtgtctgggtccctgggccagagggtcaccatctcctgca | 19 |
| | ctggaagcagctccaatgttggttatggcaattatgtgggctggtaccagcagctcccaggaacagg | |
| | ccccagaaccctcatctatcgtagtagtagccgaccctcgggggtccctgatcgattctctggctcca | |
| | ggtcaggcagcacagcaaccctgaccatctctgggctccaggctgaggatgaagccgattattactg | |
| | ctcatcctatgacagcagtctcagtggtgg | |
| IGLV 1-58 | cagtctgtgctgactcagccggcctcagtgtctgggtccctgggccagagggtcaccatctcctgca | 20 |
| | ctggaagcagctccaacatcggtagatatagtgttggctggttccagcagctcccgggaaaaggcc | |
| | ccagaaccgtcatctatagtagtagtaaccgaccctcaggggtccctgatcgattctctggctccaagt | |
| | caggcagcacagccaccctgaccatctctgggctccaggctgaggacgaggctgattattactgctc | |
| | aacatacgacagcagtctcagtagtag | |
| IGLV 1-100 | cagtctgtgctgactcagccgacctcagtgtcggggtcccttggccagagggtcaccatctcctgctc | 21 |
| | tggaagcacgaacaacatcggtattgttggtgcgagctggtaccaacagctcccaggaaaggcccc | |
| | taaactcctcgtggacagtgatggggatcgaccgtcaggggtccctgaccggttttccggctccaag | |
| | tctggcaactcagccaccctgaccatcactgggcttcaggctgaggacgaggctgattattactgcca | |
| | gtcctttgataccacgcttgatgctca | |
| IGLV 1-46 | cagtctgtgctgactcaaccagcctcagtgtccgggtctctgggccagagggtcaccatctcctgca | 22 |
| | ctggaagcagctccaacattggtagagattatgtgggctggtaccaacagctcccgggaacacgcc | |
| | ccagaaccctcatctatggtaatagtaaccgaccctcgggggtccccgatcgattctctggctccaag | |
| | tcaggcagcacagccaccctgaccatctctgggctccaggctgaggacgaggctgattattactgct | |
| | ctacatgggacaacagtctcactgttcc | |
| IGLV 3-21 | tcctatgagctgactcagccaccatccgtgaatgtgaccctgagggagacggcccacatcacctgtg | 23 |
| | ggggagacagcattggaagtaaatatgttcaatggatccagcagaatccaggccaggcccccgtgg | |
| | tgattatctataaagatagcaacaggccgacagggatccctgagcgattctctggcgccaactcagg | |
| | gaacacggctaccctgaccatcagtggggccctggccgaagacgaggctgactattactgccaggt | |
| | gggggacagtggtactaaggct | |
| IGLV 1-103 | caggctgtgctgactcagccaccctctgtgtctgcagccctggggcagagggtcaccatctcctgca | 24 |
| | ctggaagtaacaccaacatcggcagtggttatgatgtacaatggtaccagcagctcccaggaaagtc | |
| | ccctaaaactatcatttatggtaatagcaatcgaccctcgggggtcccggttcgattctctggctccaa | |
| | gtcaggcagcacagccaccctgaccatcactgggatccaggctgaggatgaggctgattattactgc | |
| | cagtcctatgatgacaacctcgatggtca | |
| IGLV 1-84 | caggctgtgctgactcagccggcctcagtgtctgggtccctgggccagagggtcaccatctcctgca | 25 |
| | ctggaagcagctccaatgttggttatggcaattatgtgggctggtaccagcagctcccaggaacagg | |
| | ccccagaaccctcatctatggtagtagttaccgaccctcgggggtccctgatcgattctctggctcca | |
| | gttcaggcagctcagccacactgaccatctctgggctccaggctgaggatgaagctgattattactgc | |
| | tcatcctatgacagcagtctcagtggtgg | |
| KAPPA V | | |
| IGKV 2-8 | gatattgtcatgacacagaccccaccgtccctgtccgtcagccctggagagccggcctccatctcct | 26 |
| | gcaaggccagtcagagcctcctgcacagtaacgggaacacctatttgaattggttccgacagaagc | |
| | caggccagtctccacagggcctgatctatagggtgtccaaccgctccactggcgtgtcagacaggtt | |
| | cagtggcagcgggtcagggacagatttcaccctgagaatcagcagagtggaggctgacgatgctg | |
| | gagtttattactgcgggcaaggtatacaagatcctcc | |
| IGKV2-11 | gatatcgtcatgacacagaccccactgtccctgtccgtcagccctggagagactgcctccatctcctg | 27 |
| | caaggccagtcagagcctcctgcacagtaacgggaacacctatttgttttggttccgacagaagcca | |
| | ggccagtctccacagcgcctgatcaacttggtttccaacagagaccctggggtcccacacaggttca | |
| | gtggcagcgggtcagggacagatttcaccctgagaatcagcagagtggaggctgacgatgctgga | |
| | gtttattactgcgggcaaggtatacaagctcctcc | |
| IGKV2-5 | gatattgtcatgacacagaccccactgtccctgtccgtcagccctggagagccggcctccatctcctg | 28 |
| | caaggccagtcagagcctcctgcacagtaatgggaacacctatttgtattggttccgacagaagcca | |
| | ggccagtctccacagcgtttgatctataaggtctccaacagagaccctggggtcccagacaggttca | |
| | gtggcagcgggtcagggacagatttcaccctgagaatcagcagagtggaggctgatgatgctgga | |
| | gtttattactgcgggcaaggtatacaagatcctcc | |
| IGKV2-4 | gatattgtcatgacacagacgccaccgtccctgtctgtcagccctagagagacggcctccatctcctg | 29 |
| | caaggccagtcagagcctcctgcacagtgatggaaacacctatttggattggtacctgcaaaagcca | |
| | ggccagtctccacagcttctgatctacttggtttccaaccgcttcactggcgtgtcagacaggttcagt | |
| | ggcagcgggtcagggacagatttcaccctgagaatcagcagagtggaggctaacgatactggagtt | |
| | tattactgcgggcaaggtacacagcttcctcc | |
| IGKV2-7 | gatattgtcatgacacagaacccactgtccctgtccgtcagccctggagagacggcctccatctcctg | 30 |
| | caaggccagtcagagcctcctgcacagtaacgggaacacctatttgaattggttccgacagaagcca | |
| | ggccagtctccacagggcctgatctataaggtctccaacagag | |

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, ABAB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention.

The present invention is described in more detail in the following non limiting Examples.

### EXAMPLES

### Example 1: Analysis of Healthy Dog PBMCs, Sorting and 10× Immune Profiling of Intact Single B Cells to Generate VH:VL Pairing Data

Two blood samples from healthy beagles were obtained from Envigo and PBMCs were isolated using Ficoll-Paque (Cytiva) density gradient centrifugation.

The cellular fraction was enriched for viable lymphocyte cells (including B cells) using FACS sorting of the forward scatter-side scatter lymphocyte gate after gating out dead cells that were stained with a viability dye. Sample 1 generated approximately 125,000 cells, while sample 2 generated approximately 200,000 cells. The resulting cells were pelleted by centrifugation and processed using the microfluidics encapsulation protocol from the 10× Genomics Chromium Next Gem Single Cell 5′ v1.1 kit (10× Genomics), following the manufacturer's protocol. The resulting cDNA was quality controlled by quantitation using the Qubit fluorometer and bioanaylser analysis (ThermoFisher). Following this, the cDNA was processed to generate 5′ VDJ NGS sequencing libraries using dog constant-region specific PCR primers as set out in Table 2 designed to cross react with as many constant regions as possible. 200 recovered 10× V-J paired cells were recovered for sample 1 and 2695 for sample 2, where IgM primers were also included.

The 5′VDJ libraries thus generated were sequenced on an Illumina NGS platform using 150 bp paired-end sequencing. With this read length, a minimum of two thousand read pairs are recommended per cell barcode. The resulting sequencing data was demultiplexed and each library will contain a Read1 and Read2.fastq.gz pair of files.

The resulting set of FASTQ files was processed via the Cell Ranger software (10× Genomics): for the combination of VDJ and 5′GEX libraries, it was processed via ‘cell ranger multi’ command, given the list of heavy and light V+D+J+C reference sequences in the dog repertoire, supplied as reference sets to the CanFam3.1 dog genome reference and annotation, including the list of validated Ig genes in the 3 IG loci in the dog genome from internal curation, as well as the list of inner primers designed for this purpose as a parameter. The results of the VDJ library through the ‘cellranger multi’ step were QCed and compared with the estimated number of cells in the cell counting step. The reconstructed chains from the Cellranger (all_contig.fasta) were blasted against the set of heavy and light V+D+J reference sequences formatted for running enclone software (10× Genomics). The results of the enclone step were sorted by aa % and filtered for the v_name sequence corresponding to the highly confident heavy+light V repertoire cell barcode calls, with only the highly confident cells chosen as the final result.

### Results:

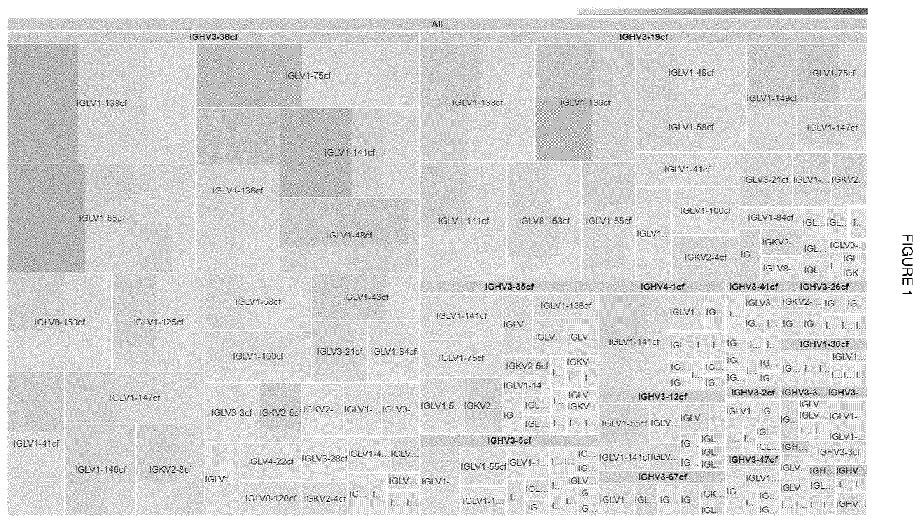

Heavy chain usage was plotted vs light chain usage from the heavy+light paired data obtained, in order to generate Treemaps (https://en.wikipedia.org/wiki/Treemapping). These are shown in FIG. 1 where blue/gray scale depicts frequency of heavy chain/light chain pairings. Some heavy/light chain pairing patterns appear more predominantly than others.

FIG. 1 shows Combined QC1 (Sample 1)+QC2-1+QC2-2 (Sample 2). Table 1a-c shows the combined data in which pairings are ranked. The top 80-90% pairings are listed. Table 3 shows the ranking of light chain V gene segments by number of different heavy V gene segment pairings.

TABLE 1a

| Heavy Chain | Light Chain | Frequency |
|---|---|---|
| IGHV3-38cf | IGLV1-138cf | 29 |
| IGHV3-38cf | IGLV1-55cf | 28 |
| IGHV3-19cf | IGLV1-138cf | 27 |
| IGHV3-38cf | IGLV1-48cf | 27 |
| IGHV3-38cf | IGLV1-125cf | 25 |
| IGHV3-38cf | IGLV1-75cf | 25 |
| IGHV3-19cf | IGLV1-136cf | 24 |
| IGHV3-19cf | IGLV8-153cf | 24 |
| IGHV3-38cf | IGLV1-136cf | 24 |
| IGHV3-38cf | IGKV2-8cf | 23 |
| IGHV3-19cf | IGLV1-141cf | 21 |
| IGHV3-38cf | IGLV1-141cf | 21 |
| IGHV3-19cf | IGLV1-147cf | 20 |
| IGHV3-19cf | IGLV1-48cf | 20 |
| IGHV3-38cf | IGLV8-153cf | 20 |
| IGHV3-38cf | IGLV1-58cf | 19 |
| IGHV3-19cf | IGLV1-149cf | 18 |
| IGHV3-35cf | IGLV1-75cf | 18 |
| IGHV3-38cf | IGLV1-100cf | 18 |
| IGHV3-38cf | IGLV1-41cf | 18 |
| IGHV3-38cf | IGLV1-147cf | 17 |
| IGHV4-1cf | IGLV1-141cf | 17 |
| IGHV3-19cf | IGKV2-8cf | 16 |
| IGHV3-19cf | IGLV1-55cf | 16 |
| IGHV3-38cf | IGLV1-46cf | 16 |
| IGHV3-38cf | IGLV3-3cf | 16 |
| IGHV3-19cf | IGLV1-125cf | 15 |
| IGHV3-19cf | IGLV1-58cf | 15 |
| IGHV3-38cf | IGLV1-103cf | 15 |
| IGHV3-38cf | IGLV3-28cf | 15 |
| IGHV3-35cf | IGLV1-55cf | 14 |
| IGHV3-38cf | IGKV2-5cf | 14 |
| IGHV3-38cf | IGLV4-22cf | 14 |
| IGHV3-19cf | IGLV1-100cf | 13 |
| IGHV3-19cf | IGLV1-41cf | 13 |
| IGHV3-19cf | IGLV1-75cf | 13 |
| IGHV3-19cf | IGLV3-21cf | 13 |
| IGHV3-19cf | IGLV8-128cf | 13 |
| IGHV3-38cf | IGKV2-11cf | 13 |
| IGHV3-38cf | IGLV1-149cf | 13 |
| IGHV3-38cf | IGLV1-84cf | 13 |
| IGHV3-38cf | IGLV3-14cf | 13 |
| IGHV3-38cf | IGLV8-128cf | 13 |
| IGHV3-5cf | IGLV1-55cf | 13 |
| IGHV3-35cf | IGKV2-8cf | 12 |
| IGHV3-35cf | IGLV1-136cf | 12 |
| IGHV3-38cf | IGKV2-4cf | 12 |
| IGHV3-19cf | IGLV1-84cf | 11 |
| IGHV3-19cf | IGLV3-27cf | 11 |
| IGHV3-38cf | IGLV1-155cf | 11 |
| IGHV3-38cf | IGLV3-21cf | 11 |
| IGHV3-38cf | IGLV3-8cf | 11 |
| IGHV3-5cf | IGLV1-141cf | 11 |
| IGHV3-19cf | IGLV1-44cf | 10 |
| IGHV3-19cf | IGLV3-28cf | 10 |
| IGHV3-26cf | IGLV1-55cf | 10 |
| IGHV3-35cf | IGLV1-141cf | 10 |
| IGHV3-35cf | IGLV1-48cf | 10 |
| IGHV3-35cf | IGLV3-21cf | 10 |
| IGHV3-35cf | IGLV8-153cf | 10 |
| IGHV3-41cf | IGLV1-138cf | 10 |
| IGHV3-5cf | IGLV1-48cf | 10 |
| IGHV4-1cf | IGLV1-138cf | 10 |
| IGHV1-30cf | IGLV1-138cf | 9 |
| IGHV3-19cf | IGLV1-46cf | 9 |
| IGHV3-35cf | IGLV1-103cf | 9 |
| IGHV3-35cf | IGLV1-138cf | 9 |
| IGHV3-38cf | IGLV1-44cf | 9 |
| IGHV3-41cf | IGLV1-136cf | 9 |
| IGHV3-41cf | IGLV1-55cf | 9 |
| IGHV3-47cf | IGLV1-48cf | 9 |
| IGHV3-67cf | IGLV1-55cf | 9 |
| IGHV4-1cf | IGLV1-48cf | 9 |
| IGHV3-19cf | IGKV2-7cf | 8 |
| IGHV3-19cf | IGLV1-103cf | 8 |
| IGHV3-26cf | IGKV2-11cf | 8 |
| IGHV3-26cf | IGLV1-138cf | 8 |
| IGHV3-26cf | IGLV1-58cf | 8 |
| IGHV3-26cf | IGLV1-75cf | 8 |
| IGHV3-26cf | IGLV8-128cf | 8 |
| IGHV3-35cf | IGLV1-100cf | 8 |
| IGHV3-35cf | IGLV1-149cf | 8 |
| IGHV3-35cf | IGLV1-41cf | 8 |
| IGHV3-35cf | IGLV3-27cf | 8 |
| IGHV3-5cf | IGLV1-136cf | 8 |
| IGHV3-8cf | IGLV1-138cf | 8 |
| IGHV3-19cf | IGKV2-4cf | 7 |
| IGHV3-19cf | IGKV2-5cf | 7 |
| IGHV3-19cf | IGLV1-82cf | 7 |
| IGHV3-19cf | IGLV4-22cf | 7 |
| IGHV3-26cf | IGKV2-4cf | 7 |
| IGHV3-26cf | IGLV8-153cf | 7 |
| IGHV3-2cf | IGLV1-141cf | 7 |
| IGHV3-35cf | IGLV1-125cf | 7 |
| IGHV3-35cf | IGLV1-147cf | 7 |
| IGHV3-35cf | IGLV1-46cf | 7 |
| IGHV3-35cf | IGLV1-58cf | 7 |
| IGHV3-35cf | IGLV3-28cf | 7 |
| IGHV3-35cf | IGLV8-128cf | 7 |
| IGHV3-38cf | IGKV2-7cf | 7 |
| IGHV3-38cf | IGLV3-27cf | 7 |
| IGHV3-5cf | IGLV1-100cf | 7 |
| IGHV3-5cf | IGLV1-103cf | 7 |
| IGHV3-5cf | IGLV1-125cf | 7 |
| IGHV3-5cf | IGLV1-46cf | 7 |
| IGHV3-5cf | IGLV8-153cf | 7 |
| IGHV3-67cf | IGKV2-8cf | 7 |
| IGHV4-1cf | IGLV1-100cf | 7 |
| IGHV4-1cf | IGLV1-58cf | 7 |
| IGHV4-1cf | IGLV4-22cf | 7 |
| IGHV1-30cf | IGLV1-136cf | 6 |
| IGHV1-30cf | IGLV1-41cf | 6 |
| IGHV3-19cf | IGKV2-11cf | 6 |
| IGHV3-26cf | IGKV2-7cf | 6 |
| IGHV3-26cf | IGLV1-136cf | 6 |
| IGHV3-26cf | IGLV1-141cf | 6 |
| IGHV3-2cf | IGLV1-100cf | 6 |
| IGHV3-35cf | IGLV1-155cf | 6 |
| IGHV3-38cf | IGLV3-26cf | 6 |
| IGHV3-39cf | IGLV1-141cf | 6 |
| IGHV3-41cf | IGLV1-141cf | 6 |
| IGHV3-41cf | IGLV1-149cf | 6 |
| IGHV3-41cf | IGLV8-153cf | 6 |
| IGHV3-47cf | IGLV1-138cf | 6 |
| IGHV3-47cf | IGLV1-55cf | 6 |
| IGHV3-5cf | IGLV1-138cf | 6 |
| IGHV3-5cf | IGLV1-75cf | 6 |
| IGHV3-67cf | IGLV1-136cf | 6 |
| IGHV3-67cf | IGLV1-46cf | 6 |
| IGHV3-67cf | IGLV1-48cf | 6 |
| IGHV3-69cf | IGLV1-55cf | 6 |
| IGHV4-1cf | IGLV1-103cf | 6 |
| IGHV4-1cf | IGLV1-136cf | 6 |
| IGHV4-1cf | IGLV1-147cf | 6 |
| IGHV4-1cf | IGLV1-149cf | 6 |
| IGHV4-1cf | IGLV1-46cf | 6 |
| IGHV1-30cf | IGKV2-5cf | 5 |
| IGHV1-30cf | IGKV2-7cf | 5 |
| IGHV1-30cf | IGLV1-100cf | 5 |
| IGHV1-30cf | IGLV8-153cf | 5 |
| IGHV3-12cf | IGLV1-138cf | 5 |
| IGHV3-19cf | IGKV2-9cf | 5 |
| IGHV3-19cf | IGLV3-14cf | 5 |
| IGHV3-19cf | IGLV3-3cf | 5 |
| IGHV3-26cf | IGKV2-8cf | 5 |
| IGHV3-26cf | IGLV1-41cf | 5 |
| IGHV3-2cf | IGLV3-27cf | 5 |
| IGHV3-35cf | IGLV1-84cf | 5 |
| IGHV3-35cf | IGLV4-22cf | 5 |
| IGHV3-38cf | IGLV8-36cf | 5 |
| IGHV3-41cf | IGKV2-8cf | 5 |
| IGHV3-41cf | IGLV1-147cf | 5 |
| IGHV3-41cf | IGLV1-46cf | 5 |
| IGHV3-41cf | IGLV1-48cf | 5 |
| IGHV3-5cf | IGKV2-8cf | 5 |
| IGHV3-5cf | IGLV1-147cf | 5 |
| IGHV3-5cf | IGLV3-28cf | 5 |
| IGHV3-5cf | IGLV8-128cf | 5 |
| IGHV3-67cf | IGLV1-75cf | 5 |
| IGHV3-69cf | IGLV1-125cf | 5 |
| IGHV3-8cf | IGLV1-149cf | 5 |
| IGHV3-8cf | IGLV1-55cf | 5 |
| IGHV3-9cf | IGLV1-100cf | 5 |
| IGHV3-9cf | IGLV1-75cf | 5 |
| IGHV4-1cf | IGLV1-55cf | 5 |
| IGHV4-1cf | IGLV1-75cf | 5 |
| IGHV4-1cf | IGLV8-153cf | 5 |
| IGHV1-30cf | IGKV2-11cf | 4 |
| IGHV1-30cf | IGLV1-141cf | 4 |
| IGHV1-30cf | IGLV1-147cf | 4 |
| IGHV1-30cf | IGLV3-27cf | 4 |
| IGHV3-12cf | IGLV1-48cf | 4 |
| IGHV3-12cf | IGLV1-55cf | 4 |
| IGHV3-19cf | IGLV1-87cf | 4 |
| IGHV3-19cf | IGLV3-11cf | 4 |
| IGHV3-26cf | IGKV2-6cf | 4 |
| IGHV3-26cf | IGLV1-100cf | 4 |
| IGHV3-26cf | IGLV1-106cf | 4 |
| IGHV3-26cf | IGLV1-149cf | 4 |
| IGHV3-26cf | IGLV1-46cf | 4 |
| IGHV3-26cf | IGLV1-50cf | 4 |
| IGHV3-26cf | IGLV1-84cf | 4 |
| IGHV3-2cf | IGLV8-153cf | 4 |
| IGHV3-32cf | IGLV1-136cf | 4 |
| IGHV3-32cf | IGLV1-138cf | 4 |
| IGHV3-32cf | IGLV1-55cf | 4 |
| IGHV3-32cf | IGLV1-75cf | 4 |
| IGHV3-35cf | IGKV2-11cf | 4 |
| IGHV3-35cf | IGLV3-14cf | 4 |
| IGHV3-38cf | IGKV2-6cf | 4 |
| IGHV3-38cf | IGKV2-9cf | 4 |
| IGHV3-38cf | IGLV1-151cf | 4 |
| IGHV3-38cf | IGLV1-162cf | 4 |
| IGHV3-38cf | IGLV1-50cf | 4 |
| IGHV3-38cf | IGLV1-82cf | 4 |
| IGHV3-38cf | IGLV3-11cf | 4 |
| IGHV3-38cf | IGLV3-24cf | 4 |
| IGHV3-38cf | IGLV4-6cf | 4 |
| IGHV3-3cf | IGLV1-103cf | 4 |
| IGHV3-41cf | IGLV1-75cf | 4 |
| IGHV3-41cf | IGLV3-14cf | 4 |
| IGHV3-41cf | IGLV3-21cf | 4 |
| IGHV3-41cf | IGLV3-3cf | 4 |
| IGHV3-41cf | IGLV4-22cf | 4 |
| IGHV3-47cf | IGLV1-141cf | 4 |
| IGHV3-47cf | IGLV1-46cf | 4 |
| IGHV3-47cf | IGLV1-75cf | 4 |
| IGHV3-47cf | IGLV3-21cf | 4 |
| IGHV3-47cf | IGLV4-22cf | 4 |
| IGHV3-50cf | IGLV1-147cf | 4 |
| IGHV3-50cf | IGLV8-153cf | 4 |
| IGHV3-5cf | IGLV1-41cf | 4 |
| IGHV3-5cf | IGLV1-44cf | 4 |
| IGHV3-5cf | IGLV1-58cf | 4 |
| IGHV3-5cf | IGLV1-84cf | 4 |
| IGHV3-67cf | IGLV1-125cf | 4 |
| IGHV3-67cf | IGLV1-141cf | 4 |
| IGHV3-67cf | IGLV3-21cf | 4 |
| IGHV3-69cf | IGKV2-8cf | 4 |
| IGHV3-8cf | IGLV1-136cf | 4 |
| IGHV3-9cf | IGLV1-136cf | 4 |
| IGHV3-9cf | IGLV1-141cf | 4 |
| IGHV4-1cf | IGLV1-155cf | 4 |
| IGHV4-1cf | IGLV1-41cf | 4 |
| IGHV4-1cf | IGLV1-44cf | 4 |
| IGHV4-1cf | IGLV3-14cf | 4 |
| IGHV4-1cf | IGLV3-28cf | 4 |
| IGHV1-30cf | IGKV2-4cf | 3 |
| IGHV1-30cf | IGLV1-103cf | 3 |
| IGHV1-30cf | IGLV1-125cf | 3 |
| IGHV1-30cf | IGLV1-44cf | 3 |
| IGHV1-30cf | IGLV1-46cf | 3 |
| IGHV1-30cf | IGLV1-48cf | 3 |
| IGHV1-30cf | IGLV1-55cf | 3 |
| IGHV1-30cf | IGLV1-75cf | 3 |
| IGHV3-12cf | IGLV1-58cf | 3 |
| IGHV3-12cf | IGLV1-75cf | 3 |
| IGHV3-16cf | IGLV1-125cf | 3 |
| IGHV3-19cf | IGLV1-155cf | 3 |
| IGHV3-19cf | IGLV1-162cf | 3 |
| IGHV3-19cf | IGLV8-36cf | 3 |
| IGHV3-26cf | IGKV2-12cf | 3 |
| IGHV3-26cf | IGKV2-9cf | 3 |
| IGHV3-26cf | IGLV1-103cf | 3 |
| IGHV3-26cf | IGLV1-147cf | 3 |
| IGHV3-26cf | IGLV4-22cf | 3 |
| IGHV3-2cf | IGKV2-11cf | 3 |
| IGHV3-2cf | IGKV2-4cf | 3 |
| IGHV3-2cf | IGLV1-136cf | 3 |
| IGHV3-2cf | IGLV1-138cf | 3 |
| IGHV3-2cf | IGLV1-147cf | 3 |
| IGHV3-2cf | IGLV1-48cf | 3 |
| IGHV3-2cf | IGLV1-55cf | 3 |
| IGHV3-2cf | IGLV1-75cf | 3 |
| IGHV3-2cf | IGLV8-36cf | 3 |
| IGHV3-32cf | IGKV2-8cf | 3 |
| IGHV3-32cf | IGLV1-141cf | 3 |
| IGHV3-32cf | IGLV8-153cf | 3 |
| IGHV3-35cf | IGKV2-5cf | 3 |
| IGHV3-35cf | IGLV1-82cf | 3 |
| IGHV3-35cf | IGLV3-11cf | 3 |
| IGHV3-35cf | IGLV3-3cf | 3 |
| IGHV3-35cf | IGLV4-6cf | 3 |
| IGHV3-38cf | IGLV1-106cf | 3 |
| IGHV3-39cf | IGKV2-11cf | 3 |
| IGHV3-39cf | IGLV1-125cf | 3 |
| IGHV3-39cf | IGLV1-46cf | 3 |
| IGHV3-3cf | IGLV1-138cf | 3 |
| IGHV3-41cf | IGLV1-100cf | 3 |
| IGHV3-41cf | IGLV3-8cf | 3 |
| IGHV3-41cf | IGLV8-128cf | 3 |
| IGHV3-47cf | IGLV1-100cf | 3 |
| IGHV3-47cf | IGLV1-103cf | 3 |
| IGHV3-47cf | IGLV1-125cf | 3 |
| IGHV3-47cf | IGLV1-136cf | 3 |
| IGHV3-47cf | IGLV1-149cf | 3 |
| IGHV3-47cf | IGLV3-27cf | 3 |
| IGHV3-47cf | IGLV4-6cf | 3 |
| IGHV3-50cf | IGLV1-138cf | 3 |
| IGHV3-5cf | IGKV2-11cf | 3 |
| IGHV3-5cf | IGKV2-5cf | 3 |
| IGHV3-5cf | IGLV1-149cf | 3 |
| IGHV3-5cf | IGLV1-155cf | 3 |
| IGHV3-5cf | IGLV1-87cf | 3 |
| IGHV3-5cf | IGLV3-11cf | 3 |
| IGHV3-5cf | IGLV3-27cf | 3 |
| IGHV3-5cf | IGLV4-22cf | 3 |
| IGHV3-67cf | IGKV2-11cf | 3 |
| IGHV3-67cf | IGLV1-100cf | 3 |
| IGHV3-67cf | IGLV1-103cf | 3 |
| IGHV3-67cf | IGLV1-149cf | 3 |
| IGHV3-69cf | IGLV1-41cf | 3 |
| IGHV3-69cf | IGLV8-153cf | 3 |
| IGHV3-6cf | IGLV8-128cf | 3 |
| IGHV3-6cf | IGLV8-153cf | 3 |
| IGHV3-81cf | IGLV1-141cf | 3 |
| IGHV3-8cf | IGLV1-141cf | 3 |
| IGHV3-8cf | IGLV1-147cf | 3 |
| IGHV3-8cf | IGLV1-48cf | 3 |
| IGHV3-8cf | IGLV1-75cf | 3 |
| IGHV3-8cf | IGLV1-84cf | 3 |
| IGHV3-8cf | IGLV3-21cf | 3 |
| IGHV3-9cf | IGLV1-125cf | 3 |
| IGHV3-9cf | IGLV1-138cf | 3 |
| IGHV3-9cf | IGLV1-55cf | 3 |
| IGHV3-9cf | IGLV8-153cf | 3 |
| IGHV4-1cf | IGKV2-7cf | 3 |
| IGHV4-1cf | IGLV1-125cf | 3 |
| IGHV4-1cf | IGLV1-82cf | 3 |
| IGHV4-1cf | IGLV1-84cf | 3 |
| IGHV4-1cf | IGLV3-21cf | 3 |
| IGHV1-30cf | IGLV1-149cf | 2 |
| IGHV1-30cf | IGLV1-84cf | 2 |
| IGHV1-30cf | IGLV3-14cf | 2 |
| IGHV1-30cf | IGLV3-28cf | 2 |
| IGHV3-12cf | IGKV2-4cf | 2 |
| IGHV3-12cf | IGLV1-100cf | 2 |
| IGHV3-12cf | IGLV1-141cf | 2 |
| IGHV3-12cf | IGLV1-147cf | 2 |
| IGHV3-12cf | IGLV1-155cf | 2 |
| IGHV3-12cf | IGLV1-41cf | 2 |
| IGHV3-12cf | IGLV3-21cf | 2 |
| IGHV3-12cf | IGLV8-128cf | 2 |
| IGHV3-12cf | IGLV8-153cf | 2 |
| IGHV3-16cf | IGLV1-141cf | 2 |
| IGHV3-16cf | IGLV1-55cf | 2 |
| IGHV3-18cf | IGLV1-149cf | 2 |
| IGHV3-18cf | IGLV1-55cf | 2 |
| IGHV3-18cf | IGLV3-28cf | 2 |
| IGHV3-18cf | IGLV8-128cf | 2 |
| IGHV3-19cf | IGKV2-6cf | 2 |
| IGHV3-19cf | IGLV1-106cf | 2 |
| IGHV3-19cf | IGLV1-73cf | 2 |
| IGHV3-19cf | IGLV4-6cf | 2 |
| IGHV3-26cf | IGLV1-125cf | 2 |
| IGHV3-26cf | IGLV1-44cf | 2 |
| IGHV3-26cf | IGLV1-82cf | 2 |
| IGHV3-2cf | IGKV2-5cf | 2 |
| IGHV3-2cf | IGKV2-7cf | 2 |
| IGHV3-2cf | IGLV1-149cf | 2 |
| IGHV3-2cf | IGLV1-41cf | 2 |
| IGHV3-2cf | IGLV1-58cf | 2 |
| IGHV3-2cf | IGLV3-14cf | 2 |
| IGHV3-2cf | IGLV3-21cf | 2 |
| IGHV3-2cf | IGLV3-28cf | 2 |
| IGHV3-2cf | IGLV4-22cf | 2 |
| IGHV3-2cf | IGLV8-128cf | 2 |
| IGHV3-32cf | IGLV1-103cf | 2 |
| IGHV3-32cf | IGLV1-58cf | 2 |
| IGHV3-32cf | IGLV3-21cf | 2 |
| IGHV3-32cf | IGLV3-27cf | 2 |
| IGHV3-32cf | IGLV3-3cf | 2 |
| IGHV3-35cf | IGKV2-16cf | 2 |
| IGHV3-35cf | IGKV2-7cf | 2 |
| IGHV3-35cf | IGLV3-24cf | 2 |
| IGHV3-35cf | IGLV3-26cf | 2 |
| IGHV3-38cf | IGLV1-49cf | 2 |
| IGHV3-38cf | IGLV1-73cf | 2 |
| IGHV3-39cf | IGLV1-100cf | 2 |
| IGHV3-39cf | IGLV1-58cf | 2 |
| IGHV3-3cf | IGKV2-11cf | 2 |
| IGHV3-41cf | IGKV2-4cf | 2 |
| IGHV3-41cf | IGKV2-7cf | 2 |
| IGHV3-41cf | IGLV1-125cf | 2 |
| IGHV3-41cf | IGLV1-155cf | 2 |
| IGHV3-41cf | IGLV1-41cf | 2 |
| IGHV3-41cf | IGLV1-49cf | 2 |
| IGHV3-41cf | IGLV1-58cf | 2 |
| IGHV3-41cf | IGLV1-84cf | 2 |
| IGHV3-41cf | IGLV3-27cf | 2 |
| IGHV3-44cf | IGKV2-8cf | 2 |
| IGHV3-44cf | IGLV1-84cf | 2 |
| IGHV3-47cf | IGKV2-4cf | 2 |
| IGHV3-47cf | IGLV1-41cf | 2 |
| IGHV3-47cf | IGLV1-44cf | 2 |
| IGHV3-47cf | IGLV8-128cf | 2 |
| IGHV3-47cf | IGLV8-153cf | 2 |
| IGHV3-50cf | IGLV1-100cf | 2 |
| IGHV3-50cf | IGLV1-48cf | 2 |
| IGHV3-54cf | IGLV1-103cf | 2 |
| IGHV3-5cf | IGLV1-106cf | 2 |
| IGHV3-5cf | IGLV1-82cf | 2 |
| IGHV3-5cf | IGLV3-14cf | 2 |
| IGHV3-5cf | IGLV3-21cf | 2 |
| IGHV3-5cf | IGLV3-8cf | 2 |
| IGHV3-67cf | IGKV2-7cf | 2 |
| IGHV3-67cf | IGLV1-147cf | 2 |
| IGHV3-67cf | IGLV1-41cf | 2 |
| IGHV3-67cf | IGLV8-128cf | 2 |
| IGHV3-67cf | IGLV8-153cf | 2 |
| IGHV3-69cf | IGKV2-7cf | 2 |
| IGHV3-69cf | IGLV1-138cf | 2 |
| IGHV3-69cf | IGLV1-141cf | 2 |
| IGHV3-69cf | IGLV1-147cf | 2 |
| IGHV3-69cf | IGLV1-48cf | 2 |
| IGHV3-69cf | IGLV1-75cf | 2 |
| IGHV3-69cf | IGLV3-21cf | 2 |
| IGHV3-6cf | IGKV2-4cf | 2 |
| IGHV3-6cf | IGKV2-7cf | 2 |
| IGHV3-6cf | IGKV2-8cf | 2 |
| IGHV3-6cf | IGLV1-136cf | 2 |
| IGHV3-6cf | IGLV1-138cf | 2 |
| IGHV3-6cf | IGLV1-141cf | 2 |
| IGHV3-6cf | IGLV1-147cf | 2 |
| IGHV3-6cf | IGLV1-155cf | 2 |
| IGHV3-6cf | IGLV1-48cf | 2 |
| IGHV3-6cf | IGLV1-75cf | 2 |
| IGHV3-6cf | IGLV3-14cf | 2 |
| IGHV3-6cf | IGLV3-21cf | 2 |
| IGHV3-6cf | IGLV3-27cf | 2 |
| IGHV3-81cf | IGKV2-8cf | 2 |
| IGHV3-81cf | IGLV1-103cf | 2 |
| IGHV3-81cf | IGLV1-125cf | 2 |
| IGHV3-81cf | IGLV1-48cf | 2 |
| IGHV3-81cf | IGLV1-55cf | 2 |
| IGHV3-81cf | IGLV1-75cf | 2 |
| IGHV3-8cf | IGKV2-4cf | 2 |
| IGHV3-8cf | IGLV1-46cf | 2 |
| IGHV3-8cf | IGLV1-58cf | 2 |
| IGHV3-8cf | IGLV3-14cf | 2 |
| IGHV3-9cf | IGKV2-5cf | 2 |
| IGHV3-9cf | IGKV2-6cf | 2 |
| IGHV3-9cf | IGKV2-7cf | 2 |
| IGHV3-9cf | IGLV1-41cf | 2 |
| IGHV3-9cf | IGLV1-46cf | 2 |
| IGHV3-9cf | IGLV1-58cf | 2 |
| IGHV3-9cf | IGLV3-3cf | 2 |
| IGHV3-9cf | IGLV4-22cf | 2 |
| IGHV3-9cf | IGLV8-128cf | 2 |
| IGHV4-1cf | IGKV2-12cf | 2 |
| IGHV4-1cf | IGKV2-16cf | 2 |
| IGHV4-1cf | IGKV2-4cf | 2 |
| IGHV4-1cf | IGKV2-5cf | 2 |
| IGHV4-1cf | IGLV3-11cf | 2 |
| IGHV4-1cf | IGLV3-8cf | 2 |
| IGHV4-1cf | IGLV8-128cf | 2 |
| IGHV1-17cf | IGLV1-138cf | 1 |
| IGHV1-30cf | IGLV1-151cf | 1 |
| IGHV1-30cf | IGLV1-49cf | 1 |
| IGHV1-30cf | IGLV1-58cf | 1 |
| IGHV1-30cf | IGLV1-70cf | 1 |
| IGHV1-30cf | IGLV1-87cf | 1 |
| IGHV1-30cf | IGLV3-11cf | 1 |
| IGHV1-30cf | IGLV3-21cf | 1 |
| IGHV1-30cf | IGLV3-24cf | 1 |
| IGHV1-30cf | IGLV4-6cf | 1 |
| IGHV1-30cf | IGLV8-36cf | 1 |
| IGHV3-12cf | IGKV2-5cf | 1 |
| IGHV3-12cf | IGKV2-7cf | 1 |
| IGHV3-12cf | IGLV1-103cf | 1 |
| IGHV3-12cf | IGLV1-125cf | 1 |
| IGHV3-12cf | IGLV1-136cf | 1 |
| IGHV3-12cf | IGLV1-149cf | 1 |
| IGHV3-12cf | IGLV1-73cf | 1 |
| IGHV3-12cf | IGLV1-84cf | 1 |
| IGHV3-12cf | IGLV1-86cf | 1 |
| IGHV3-12cf | IGLV3-27cf | 1 |
| IGHV3-12cf | IGLV3-3cf | 1 |
| IGHV3-12cf | IGLV3-8cf | 1 |
| IGHV3-12cf | IGLV4-6cf | 1 |
| IGHV3-12cf | IGLV8-36cf | 1 |
| IGHV3-16cf | IGLV1-138cf | 1 |
| IGHV3-16cf | IGLV1-147cf | 1 |
| IGHV3-16cf | IGLV1-75cf | 1 |
| IGHV3-16cf | IGLV1-86cf | 1 |
| IGHV3-16cf | IGLV3-3cf | 1 |
| IGHV3-18cf | IGLV1-103cf | 1 |
| IGHV3-18cf | IGLV1-147cf | 1 |
| IGHV3-18cf | IGLV1-48cf | 1 |
| IGHV3-18cf | IGLV1-50cf | 1 |
| IGHV3-18cf | IGLV1-82cf | 1 |
| IGHV3-18cf | IGLV3-21cf | 1 |
| IGHV3-18cf | IGLV8-153cf | 1 |
| IGHV3-19cf | IGKV2-12cf | 1 |
| IGHV3-19cf | IGKV2-16cf | 1 |
| IGHV3-19cf | IGLV1-151cf | 1 |
| IGHV3-19cf | IGLV1-50cf | 1 |
| IGHV3-19cf | IGLV1-67cf | 1 |
| IGHV3-19cf | IGLV3-24cf | 1 |
| IGHV3-19cf | IGLV3-8cf | 1 |
| IGHV3-24cf | IGLV1-147cf | 1 |
| IGHV3-24cf | IGLV1-58cf | 1 |
| IGHV3-24cf | IGLV1-75cf | 1 |
| IGHV3-24cf | IGLV3-14cf | 1 |
| IGHV3-26cf | IGKV2-5cf | 1 |
| IGHV3-26cf | IGLV3-27cf | 1 |
| IGHV3-26cf | IGLV8-36cf | 1 |
| IGHV3-27cf | IGLV1-138cf | 1 |
| IGHV3-2cf | IGKV2-6cf | 1 |
| IGHV3-2cf | IGKV2-8cf | 1 |
| IGHV3-2cf | IGKV2-9cf | 1 |
| IGHV3-2cf | IGLV1-103cf | 1 |
| IGHV3-2cf | IGLV1-125cf | 1 |
| IGHV3-2cf | IGLV1-155cf | 1 |
| IGHV3-2cf | IGLV1-44cf | 1 |
| IGHV3-2cf | IGLV1-46cf | 1 |
| IGHV3-2cf | IGLV1-84cf | 1 |
| IGHV3-2cf | IGLV3-24cf | 1 |
| IGHV3-2cf | IGLV3-26cf | 1 |
| IGHV3-2cf | IGLV3-8cf | 1 |

TABLE 1b

| Heavy Chain | Frequency |
|---|---|
| IGHV3-38cf | 2767 |
| IGHV3-19cf | 1758 |
| IGHV3-35cf | 681 |
| IGHV3-5cf | 434 |
| IGHV4-1cf | 384 |
| IGHV3-41cf | 267 |
| IGHV3-26cf | 238 |
| IGHV1-30cf | 220 |
| IGHV3-67cf | 199 |
| IGHV3-47cf | 181 |
| IGHV3-12cf | 180 |
| IGHV3-2cf | 177 |
| IGHV3-8cf | 120 |
| IGHV3-9cf | 120 |
| IGHV3-32cf | 117 |
| IGHV3-39cf | 89 |
| IGHV3-69cf | 85 |
| IGHV3-6cf | 75 |
| IGHV3-50cf | 52 |
| IGHV3-81cf | 46 |
| IGHV3-3cf | 39 |
| IGHV3-18cf | 29 |
| IGHV3-44cf | 26 |
| IGHV3-16cf | 23 |
| IGHV3-80cf | 12 |
| IGHV3-54cf | 12 |
| IGHV3-64cf | 11 |
| IGHV3-37cf | 7 |
| IGHV3-70cf | 5 |
| IGHV3-46cf | 5 |
| IGHV3-33cf | 5 |
| IGHV3-24cf | 5 |
| IGHV1-17cf | 4 |
| IGHV3-72cf | 4 |
| IGHV3-59cf | 2 |
| IGHV3-49cf | 2 |
| IGHV3-27cf | 2 |
| IGHV3-43cf | 2 |
| IGHV3-78cf | 1 |

TABLE 1c

| Light Chain | Frequency |
|---|---|
| IGLV1-138cf | 683 |
| IGLV1-141cf | 629 |
| IGLV1-55cf | 597 |
| IGLV1-136cf | 535 |
| IGLV1-75cf | 435 |
| IGLV1-48cf | 429 |
| IGLV8-153cf | 387 |
| IGLV1-147cf | 285 |
| IGLV1-125cf | 284 |
| IGLV1-41cf | 283 |
| IGKV2-8cf | 279 |
| IGLV1-149cf | 267 |
| IGLV1-58cf | 265 |
| IGLV1-100cf | 256 |
| IGLV1-46cf | 231 |
| IGLV3-21cf | 203 |
| IGLV1-103cf | 173 |
| IGLV1-84cf | 164 |
| IGLV8-128cf | 164 |
| IGKV2-11cf | 146 |
| IGKV2-5cf | 145 |
| IGLV4-22cf | 140 |
| IGLV3-27cf | 136 |
| IGKV2-4cf | 127 |
| IGLV3-28cf | 121 |
| IGKV2-7cf | 113 |
| IGLV3-14cf | 106 |
| IGLV1-44cf | 103 |
| IGLV1-155cf | 92 |
| IGLV3-3cf | 92 |
| IGLV1-82cf | 70 |
| IGLV3-8cf | 50 |
| IGLV8-36cf | 43 |
| IGLV3-11cf | 39 |
| IGKV2-6cf | 33 |
| IGLV4-6cf | 28 |
| IGKV2-9cf | 26 |
| IGLV3-24cf | 26 |
| IGLV1-162cf | 25 |
| IGLV1-106cf | 22 |
| IGLV3-26cf | 21 |
| IGLV1-49cf | 18 |
| IGLV1-50cf | 18 |
| IGLV1-87cf | 16 |
| IGLV1-73cf | 15 |
| IGLV1-151cf | 13 |
| IGKV2-16cf | 13 |
| IGKV2-12cf | 12 |
| IGLV1-86cf | 8 |
| IGLV1-67cf | 8 |
| IGLV1-70cf | 3 |
| IGLV1-132cf | 3 |
| IGLV4-12cf | 2 |
| IGLV1-112cf | 2 |
| IGKV3-18cf | 1 |
| IGLV1-72cf | 1 |

TABLE 2

| 5′-3′ primer sequence | Name | SEQ ID NO |
|---|---|---|
| Heavy-IgG | | |
| CTGGGGGCCAGTGGGAAAAC | cIgG(1-4)_IN-Short | 31 |
| GCACACCGCTGGTCAAGGAG | cIgG(1-4)_IN-Long | 32 |
| GCACTGTCACCATGCTGCTG | cIgG(1-4)_OUT | 33 |
| Heavy-IgA | | |
| CAGATGTGCTGTCCTTGCCG | cIgA_IN_Long | 34 |
| GGGTCAACTGGCTGCTCATG | cIgA_OUT | 35 |
| CAGAGGCTCAGCGGGAACAC | cIgA_IN_Short | 36 |
| Light-Lambda | | |
| GGTAGCTGCTGGCCGTGTAC | cIgL(1-9)_OUT_v3 | 37 |
| GCCCTGGATGATGGTGCTGC | cIgL(1-9)_IN_v3_Long | 38 |
| TCAGAGGAGGGCGGGAAGAG | cIgL(1-9)_IN_v3_Short | 39 |
| CTGCTTGGAGGGCTTGGTGG | cIgL(1-9)_IN_v4_Long | 40 |
| Light-Kappa | | |
| TCCTGGATGCCTGTGTCTTGG | cIgK_IN_Long | 41 |
| GGTGCTGCTGAGGCTGTAGG | cIgK_OUT | 42 |
| AAATAGACGGCTGGCTGGGC | cIgK_IN_Short | 43 |
| Heavy-IgM | | |
| GAACACCTGAGAGGTCGCCAC | cIgM_OUT | 44 |
| CCTGGTTGTTGATTGCACTGAGG | cIgM_IN-Long | 45 |
| TGATGAGGGGGAAGAGGTTTGG | cIgM_IN-Short | 46 |
| Heavy-IgD | | |
| GTTTCCAGGGGGTGGACAGG | cIgD_OUT | 47 |
| CCATCTGGGCCTGTGACTCTG | cIgD_IN-Long | 48 |
| CCTTACATCCTGAGACCAAGGGG | cIgD_IN_Short | 49 |

TABLE 3

| | Number of different |
|---|---|
| Light Chain | heavy chain pairings |
| IGLV1-138cf | 27 |
| IGLV1-136cf | 24 |
| IGLV1-141cf | 24 |
| IGLV1-48cf | 23 |
| IGLV1-55cf | 23 |
| IGLV1-103cf | 23 |
| IGLV1-41cf | 22 |
| IGLV8-153cf | 22 |
| IGLV1-75cf | 22 |
| IGLV1-147cf | 21 |
| IGLV8-128cf | 20 |
| IGLV1-58cf | 20 |
| IGLV1-100cf | 20 |
| IGLV1-125cf | 20 |
| IGLV1-84cf | 19 |
| IGLV1-46cf | 19 |
| IGKV2-8cf | 19 |
| IGLV3-21cf | 19 |
| IGLV1-149cf | 18 |
| IGLV3-14cf | 17 |
| IGKV2-7cf | 17 |
| IGLV3-27cf | 16 |
| IGKV2-5cf | 15 |
| IGKV2-4cf | 15 |
| IGLV4-22cf | 13 |
| IGLV1-82cf | 13 |
| IGLV3-28cf | 13 |
| IGKV2-11cf | 13 |
| IGLV8-36cf | 12 |
| IGLV1-44cf | 12 |
| IGLV3-11cf | 11 |
| IGLV3-8cf | 11 |
| IGLV1-155cf | 11 |
| IGLV3-3cf | 10 |
| IGLV3-24cf | 9 |
| IGLV1-49cf | 9 |
| IGKV2-6cf | 8 |
| IGLV1-73cf | 7 |
| IGLV1-162cf | 7 |
| IGLV4-6cf | 6 |
| IGLV1-106cf | 6 |
| IGLV1-50cf | 5 |
| IGLV3-26cf | 5 |
| IGKV2-12cf | 5 |
| IGLV1-151cf | 4 |
| IGKV2-9cf | 4 |
| IGKV2-16cf | 4 |
| IGLV1-67cf | 4 |
| IGLV1-86cf | 4 |
| IGLV1-87cf | 4 |
| IGLV1-70cf | 3 |
| IGLV1-132cf | 2 |
| IGKV3-18cf | 1 |
| IGLV4-12cf | 1 |
| IGLV1-112cf | 1 |
| IGLV1-72cf | 1 |

1. A rodent or rodent cell comprising a genome comprising no more than 8 dog IGHV gene segments, one or more dog D region gene segment(s) and one or more dog J region gene segment(s), wherein the rodent or rodent cell is capable of expressing the dog gene segments to form an antibody heavy chain, and wherein at least one of the IGH V gene segments is selected from the list comprising V3-38, V3-19, V3-35, V3-5, V4-1, V3-41, V3-26 and V1-30.

2. The rodent or rodent cell according to claim 1 comprising dog IGH:
(i) V3-38, optionally with up to 7 other dog IGH V gene segments,
(ii) V3-38 and V3-19, optionally with up to 6 other dog IGH V gene segments,
(iii) V3-38, V3-19, and V3-35, optionally with up to 5 other dog IGH V gene segments,
(iv) V3-38, V3-19, V3-35, and V3-5, optionally with up to 4 other dog IGH V gene segments,
(v) V3-38, V3-19, V3-35, V3-5, V4-1, optionally with up to 3 other dog IGH V gene segments,
(vi) V3-38, V3-19, V3-35, V3-5, V4-1, V3-41, optionally with up to 2 other dog IGH V gene segments,
(vii) V3-38, V3-19, V3-35, V3-5, V4-1, V3-41, V3-26, optionally with up to 1 other dog IGH V gene segment,
(viii) V3-38, V3-19, V3-35, V3-5, V4-1, V3-41, V3-26, and V1-30.

3. The rodent or rodent cell according to claim 1 comprising no more than 7, no more than 6, no more than 5, no more than 4, no more than 3, no more than two or only 1 of the dog IGH V gene segment(s), optionally wherein the rodent or rodent cell has a genome encoding only a single type of dog heavy chain VDJ variable region.

4. The rodent or rodent cell according to claim 1 comprising at least 2 IgH gene segments, such as at least 3, at least 4, at least 5, at least 6, at least 7 or 8 dog IGH V gene segments.

5. The rodent or rodent cell according to claim 1 comprising at least one gene segment from IGH V gene segment of family 1, 3 and 4.

6. A rodent or rodent cell comprising a genome comprising no more than 17 dog lambda V gene segments and one or more dog J region gene segment(s); wherein the rodent or rodent cell capable of expressing the dog lambda gene segments to form an antibody light chain, and wherein at least one of the dog lambda gene segments is selected from 1-138, 1-141, 1-55,1-136, 1-75, 1-48, 8-153, 1-147, 1-125, 1-41, 1-149, 1-58, 1-100, 1-46, 3-21, 1-103 and 1-84.

7. The rodent or rodent cell according to claim 6 comprising dog lambda V:
(i) 1-138, optionally with up to 16 other dog lambda V gene segments,
(ii) 1-138, 1-141 optionally with up to 15 other dog lambda V gene segments,
(iii) 1-138, 1-141, 1-55, optionally with up to 14 other dog lambda V gene segments,
(iv) 1-138, 1-141, 1-55, 1-136, optionally with up to 13 other dog lambda V gene segments,
(v) 1-138, 1-141, 1-55, 1-136, 1-75, optionally with up to 12 other dog lambda V gene segments,
(vi) 1-138, 1-141, 1-55, 1-136, 1-75, 1-48, optionally with up to 11 other dog lambda V gene segments
(vii) 1-138, 1-141, 1-55, 1-136, 1-75, 1-48, 8-153, optionally with up to 10 other dog lambda V gene segments.

8. The rodent or rodent cell according to claim 6 comprising no more than 16, no more than 15, no more than 14, no more than 13, no more than 12, no more than 11, no more than 10, no more than 9, no more than 8, no more than 7, no more than 6, no more than 5, no more than 4, no more than 3, no more than 2 or only 1 V gene segment, optionally wherein the rodent or rodent cell has a genome encoding only a single type of dog lambda light chain VJ variable region.

9. The rodent or rodent cell according to claim 6 comprising at least 2 dog lambda V gene segments, such as at least 3, at least 4, at least 5, at least 6, at least 7 or 8 dog lambda V gene segments.

10. The rodent or rodent cell according to claim 6 comprising a lambda V gene segment from each of the lambda V families 1, 2, 3, 4, 5 and 8.

11. A rodent or rodent cell comprising a genome comprising no more than 5 dog kappa V gene segments and one or more dog J region gene segment(s), wherein the rodent or rodent cell is capable of expressing the dog kappa gene segments to form an antibody light chain, and wherein at least one of the dog kappa gene segments is selected from 2-8, 2-11, 2-5, 2-4 and 2-7.

12. The rodent or rodent cell according to claim 11 comprising dog kappa V:
(i) 2-8, optionally with up to 4 other dog kappa V gene segments;
(ii) 2-8, 2-11 optionally with up to 3 other dog kappa V gene segments;
(iii) 2-8, 2-11, 2-5 optionally with up to 2 other dog kappa V gene segments;
(iv) 2-8, 2-11, 2-5, 2-4 optionally with up to 1 other dog kappa V gene segments; or
(v) 2-8, 2-11, 2-5, 2-4 and 2-7.

13. The rodent or rodent cell according to claim 11 comprising no more than 4, no more than 3, no more than 2, or only 1 dog kappa V gene segment, optionally wherein the rodent or rodent cell has a genome encoding only a single type of dog kappa light chain VJ variable region.

14. The rodent or rodent cell according to claim 11 comprising at least 2 dog kappa V gene segments, such as at least 3, at least 4, or 5 dog kappa V gene segments.

15. The rodent or rodent cell according to claim 11 comprising a kappa V gene segment from each of the kappa V gene segment families 2, 3 and 4, suitably including IGKV3-18 and IGKV4-15.

16. The rodent or rodent cell of any one of claim 1, 6 or 11 wherein the rodent genome comprises dog gene segments from both the heavy chain and at least one dog light chain, optionally wherein;
(i) the rodent or cell genome is according to claim 1 in combination with dog lambda gene segments according to any one of claim 6, and/or dog kappa V gene segments according to any of claim 11, or
(ii) the rodent or cell genome is according to claim 1 and additionally comprises dog kappa V and J gene segments capable of being expressed to form a kappa antibody light chain variable domain; and/or the rodent or cell genome is a rodent or rodent cell according to claim 1 additionally comprising dog lambda V and J gene segments capable of being expressed to form a lambda antibody light chain.

17. The rodent or rodent cell of claim 16 wherein:
(i) the dog gene segments(s) are located in the genome upstream of a rodent constant region, suitably upstream of the heavy chain constant region for inserted dog heavy chain variable region gene segments and/or suitably upstream of a light chain constant region for inserted dog light chain variable region gene segments, such that the rodent or rodent cell is able to produce a chimeric antibody heavy chain, or light chain, or both, resulting from expression of the inserted variable region gene segments and a host constant region; or
(ii) the dog gene segments(s) are located into the genome in functional arrangement with a dog constant region, such that the rodent is able to produce an antibody chain resulting from the expression of the dog VDJ gene segments with the dog constant region, and/or an antibody chain resulting from the expression of the dog VJ gene segments with the dog constant region.

18. The rodent or rodent cell according to any one of claim 16 comprising a genome in which one or more dog V gene segments are regulated by a non-coding regulatory sequence, which is one or more of: a promoter preceding each V gene segment coding sequence, intron, splice site, and recombination signal sequences for V(D)J recombination, wherein the regulatory sequence is of endogenous host origin.

19. (canceled)

20. An antibody chain comprising a variable region obtained or obtainable by expression of any one of dog IGH V3-38, V3-19, V3-35, V3-5, V4-1, V3-41, V3-26 and V1-30 in a rodent, in conjunction with a rodent constant region.

21. (canceled)

22. An antibody chain comprising a variable region obtained or obtainable by expression of any one of dog lambda V 1-138, 1-141, 1-55, 1-136, 1-75, 1-48, 8-153, 1-147, 1-125, 1-41, 1-149, 1-58, 1-100, 1-46, 3-21, 1-103, and 1-84 in a rodent, in conjunction with a rodent constant region.

23. (canceled)

24. An antibody chain having comprising a variable region obtained or obtainable by expression of any one of dog kappa V2-8, 2-11, 2-5, 2-4 and 2-7 in a rodent, in conjunction with a rodent constant region.

25-40. (canceled)

41. A pharmaceutical composition comprising the antibodies according to any one of claim 20, 22 or 24 and further comprising a pharmaceutically acceptable excipient or carrier.


SEQUENCE LISTING (49 sequences)

SEQ ID NO: 1 - DNA, 296 bp, Canis
gaggtgcagctggtggagtctgggggagacctggtgaagcctggggggaccttgagactgtcctgtgtggcctctggattcacctttagtagctatgacatgagctgggtccgtcagtctccagggaaggggctgcagtgggtcgcagttatttggaatgatggaagtagcacatactacgcagacgctgtgaagggccgattcaccatctccagagacaacgccaagaacacgctgtatctgcagatgaacagcctgagagccgaggacacggccgtgtattactgtgcgaagga

SEQ ID NO: 2 - DNA, 296 bp, Canis
gaggtgcagctggtggagtctgggggagacctggtgaagcctgcggggtccctgagactgtcctgtgtggcctctggattcaccttcagtagctacagcatgagctgggtccgccaggctcctgagaaggggctgcagttggtcgcaggtattaacagcggtggaagtagcacatactacacagacgctgtgaagggccgattcaccatctccagagacaacgccaagaacacagtgtatctgcagatgaacagcctgagagccgaggacacggccatgtattactgtgcaaagga

SEQ ID NO: 3 - DNA, 296 bp, Canis
gaggtgcagctggtggagtctgggggagacctggtgaagcctgtgggatccctgagactctcctgtgtggcctctggattcaccttcagtagctatgacatgaactgggtccgccaggctccagggaaggggctgcagtgggtcgcatacattagcagtggtggaagtagcacatactatgcagatgctgtgaagggccggttcaccatctccagagacaacgccaagaacacgctgtatcttcagatgaacagcctgagagccgaggacacggccatgtattactgtgcgggtga

SEQ ID NO: 4 - DNA, 296 bp, Canis
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgagtga

SEQ ID NO: 5 - DNA, 290 bp, Canis
gaactcacactgcaggagtcagggccaggactggtgaagccctcacagaccctctctctcacctgtgttgtgtccggaggctccgtcaccagcagttactactggaactggatccgccagcgccctgggaggggactggaatggatggggtactggacaggtagcacaaactacaacccggcattccagggacgcatctccatcactgctgacacggccaagaaccagttctccctgcagctgagctccatgaccaccgaggacacggccgtgtattactgtgcaagaga

SEQ ID NO: 6 - DNA, 296 bp, Canis
gaggtgcagctggtggagtctgggggagacctggtgaagcctggggggtccctgagactctcctgtgtagcctctggattcaccttcagtaactacgacatgagctgggtccgccaggctcctgggaaggggctgcagtgggtcgcagctattagctatgatggaagtagcacatactacactgacgctgtgaagggccgattcaccatctccagagacaacgccaggaacacagtgtatctgcagatgaacagcctgagagccgaggacacggctgtgtattactgtgcgaagga

SEQ ID NO: 7 - DNA, 296 bp, Canis
gaggtgtagctggtggagtctgggggagacctggtgaagcctggggggtccctgagactctcctgtgtgggctctggattcaccttcagtagctactggatgagctgggtccgccaggctccagggaaggggctacagtgggttgcagaaattagcggtagtggaagtagcacaaactatgcagacgctgtgaagggccgattcatcatctccagagacaatgccaagaacacgctgtatctgcagatgaacagcctgagagccgaggacacggccatgtattactgtgcaaggga

SEQ ID NO: 8 - DNA, 294 bp, Canis
gaggtccagctggtgcagtctggggctgaggtgaagaagccaggggcatctgtgaaggtctcctgcaagacatctggatacaccttcattaactactatatgatctgggtacgacaggctccaggagcagggcttgattggatgggacagattgatcctgaagatggtgccacaagttatgcacagaagttccagggcagagtcaccctgacagcagacacatccacaagcacagcctacatggagctgagcagtctgagagctggggacatagctgtgtactactgtgcgaga

SEQ ID NO: 9 - DNA, 299 bp, Canis
cagtctgtgctgactcagccggcctcagtgtctgggtccctgggccagagggtcaccatctcctgcactggaagcagctccaatgttggttatggcaattatgtgggctggtaccagcagctcccaggaacaagccccagaaccctcatctatgatagtagtagccgaccctcgggggtccctgatcgattctctggctccaggtcaggcagcacagcaaccctgaccatctctgggctccaggctgaggatgaagccgattattactgctcatcctatgacagcagtctcagtggtgg

SEQ ID NO: 10 - DNA, 296 bp, Canis
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtgtacagtgttggggatcgaccgtcaggggtccctgaccggttttccggctccaactctggcaactcagccaccctgaccatcactgggcttcaggctgaggacgaggctgattattactgccagtcctttgataccacgcttggtgctca

SEQ ID NO: 11 - DNA, 296 bp, Canis
cagtctgtgctgactcagccggcctcagtgtctgggtccctgggccagaggatcaccatctcctgcactggaagcagctccaacattggaggtaataatgtgggttggtaccagcagctcccaggaagaggccccagaactgtcatctatagtacaaatagtcgaccctcgggggtgcccgatcgattctctggctccaagtctggcagcacagccaccctgaccatctctgggctccaggctgaggatgaggctgattattactgctcaacgtgggatgatagtctcagtgctcc

SEQ ID NO: 12 - DNA, 296 bp, Canis
cagtctgtgctgactcagccggcctcagtgtctgggtccctgggccagagggtcaccatctcctgcactggaagcagctccaacatcggtagaggttatgtgggctggtaccagcagctcccaggaacaggccccagaaccctcatctatgatagtagtagccgaccctcgggggtccctgatcgattctctggctccaggtcaggcagcacagcaaccctgaccatctctgggctccaggctgaggacgaggctgattattactgctcagcatatgacagcagtctcagtggtgg

SEQ ID NO: 13 - DNA, 296 bp, Canis
cagtctgtgctgactcagccggcctcagtgactgggtccctgggccagagggtcaccatctcctgcactggaagcagctccaacatcggtggatataatgttggctggttccagcagctcccgggaacaggccccagaaccgtcatctatagtagtagtaaccgaccctcgggggtcccggatcgattctctggctccaggtcaggcagcacagccaccctgaccatctctgggctccaggctgaggacgaggctgagtattactgctcaacatgggacagcagtctcaaagctcc

SEQ ID NO: 14 - DNA, 296 bp, Canis
cagtctatgctgactcagccagcctcagtgtctgggtccctgggccagaaggtcaccatctcctgcactggaagcagctccaacatcggtggtaattatgtgggctggtaccaacagctcccaggaataggccctagaaccgtcatctatggtaataattaccgaccttcaggggtccccgatcgattctctggctccaagtcaggcagttcagccaccctgaccatctctgggctccaggctgaggacgaggctgagtattactgctcatcatgggatgatagtctcagaggtca

SEQ ID NO: 15 - DNA, 296 bp, Canis
cagactgtggtaacccaggagccatcactctcagtgtctccaggagggacagtcacactcacatgtggcctcagctctgggtcagtctctacaagtaattaccctggctggtaccagcagacccaaggccgggctcctcgcacgattatctacaacacaagcagccgcccctctggggtccctaatcgcttctctggatccatctctggaaacaaagccgccctcaccatcacaggagcccagcccgaggatgaggctgactattactgttccttgtatacgggtagttacactga

SEQ ID NO: 16 - DNA, 296 bp, Canis
cagtctgtgctgactcagccggcctcagtgtctgggtccctgggccagagggtcaccatctcctgcactggaagcagctccaacatcggtagaggttatgtgggctggtaccagcagctcccaggaacaggccccagaaccctcatctatgataatagtaaccgaccctcgggggtccctgatcgattctctggctccaagtcaggcagcacagccaccctgaccatctctgggctccaggctgaggacgaggctgattattactgctcaacatacgacagcagtctcagtggtgg

SEQ ID NO: 17 - DNA, 296 bp, Canis
cagtctgtgctgactcagccggcctcagtgtccgggtccctgggccagagggtcaccatctcctgcactggaagcagctccaacatcggtagaggttatgtgggctggtaccaacagctcccgggaacaggccccagaaccctcatctatggtaatagtaaccgaccctcaggggtccccgatcggttctctggctccaggtcaggcagcacagccaccctgaccatctctgggctccaggctgaggatgaggctgattattactgctcatcgtgggacagcagtctcagtgctct

SEQ ID NO: 18 - DNA, 299 bp, Canis
cagtctgtgctgactcagccagcctccgtgtctgggtccctgggccagagggtcaccatttcctgcactggaagcagctccaacgttggttatagcagtagtgtgggctggtaccagcagttcccaggaacaggccccagaaccatcatctattatgatagtagccgaccctcgggggtccccgatcgattctctggctccaagtctggcagcacagccaccctgaccatctctgggctccaggctgaggatgaggctgattattactgctcatcttgggacaacagtctcaaagctcc

SEQ ID NO: 19 - DNA, 299 bp, Canis
cagtctgtgctgactcagccggcctcagtgtctgggtccctgggccagagggtcaccatctcctgcactggaagcagctccaatgttggttatggcaattatgtgggctggtaccagcagctcccaggaacaggccccagaaccctcatctatcgtagtagtagccgaccctcgggggtccctgatcgattctctggctccaggtcaggcagcacagcaaccctgaccatctctgggctccaggctgaggatgaagccgattattactgctcatcctatgacagcagtctcagtggtgg

SEQ ID NO: 20 - DNA, 296 bp, Canis
cagtctgtgctgactcagccggcctcagtgtctgggtccctgggccagagggtcaccatctcctgcactggaagcagctccaacatcggtagatatagtgttggctggttccagcagctcccgggaaaaggccccagaaccgtcatctatagtagtagtaaccgaccctcaggggtccctgatcgattctctggctccaagtcaggcagcacagccaccctgaccatctctgggctccaggctgaggacgaggctgattattactgctcaacatacgacagcagtctcagtagtag

SEQ ID NO: 21 - DNA, 296 bp, Canis
cagtctgtgctgactcagccgacctcagtgtcggggtcccttggccagagggtcaccatctcctgctctggaagcacgaacaacatcggtattgttggtgcgagctggtaccaacagctcccaggaaaggcccctaaactcctcgtggacagtgatggggatcgaccgtcaggggtccctgaccggttttccggctccaagtctggcaactcagccaccctgaccatcactgggcttcaggctgaggacgaggctgattattactgccagtcctttgataccacgcttgatgctca

SEQ ID NO: 22 - DNA, 296 bp, Canis
cagtctgtgctgactcaaccagcctcagtgtccgggtctctgggccagagggtcaccatctcctgcactggaagcagctccaacattggtagagattatgtgggctggtaccaacagctcccgggaacacgccccagaaccctcatctatggtaatagtaaccgaccctcgggggtccccgatcgattctctggctccaagtcaggcagcacagccaccctgaccatctctgggctccaggctgaggacgaggctgattattactgctctacatgggacaacagtctcactgttcc

SEQ ID NO: 23 - DNA, 288 bp, Canis
tcctatgagctgactcagccaccatccgtgaatgtgaccctgagggagacggcccacatcacctgtgggggagacagcattggaagtaaatatgttcaatggatccagcagaatccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtactaaggct

SEQ ID NO: 24 - DNA, 299 bp, Canis
caggctgtgctgactcagccaccctctgtgtctgcagccctggggcagagggtcaccatctcctgcactggaagtaacaccaacatcggcagtggttatgatgtacaatggtaccagcagctcccaggaaagtcccctaaaactatcatttatggtaatagcaatcgaccctcgggggtcccggttcgattctctggctccaagtcaggcagcacagccaccctgaccatcactgggatccaggctgaggatgaggctgattattactgccagtcctatgatgacaacctcgatggtca

SEQ ID NO: 25 - DNA, 299 bp, Canis
caggctgtgctgactcagccggcctcagtgtctgggtccctgggccagagggtcaccatctcctgcactggaagcagctccaatgttggttatggcaattatgtgggctggtaccagcagctcccaggaacaggccccagaaccctcatctatggtagtagttaccgaccctcgggggtccctgatcgattctctggctccagttcaggcagctcagccacactgaccatctctgggctccaggctgaggatgaagctgattattactgctcatcctatgacagcagtctcagtggtgg

SEQ ID NO: 26 - DNA, 302 bp, Canis
gatattgtcatgacacagaccccaccgtccctgtccgtcagccctggagagccggcctccatctcctgcaaggccagtcagagcctcctgcacagtaacgggaacacctatttgaattggttccgacagaagccaggccagtctccacagggcctgatctatagggtgtccaaccgctccactggcgtgtcagacaggttcagtggcagcgggtcagggacagatttcaccctgagaatcagcagagtggaggctgacgatgctggagtttattactgcgggcaaggtatacaagatcctcc

SEQ ID NO: 27 - DNA, 302 bp, Canis
gatatcgtcatgacacagaccccactgtccctgtccgtcagccctggagagactgcctccatctcctgcaaggccagtcagagcctcctgcacagtaacgggaacacctatttgttttggttccgacagaagccaggccagtctccacagcgcctgatcaacttggtttccaacagagaccctggggtcccacacaggttcagtggcagcgggtcagggacagatttcaccctgagaatcagcagagtggaggctgacgatgctggagtttattactgcgggcaaggtatacaagctcctcc

SEQ ID NO: 28 - DNA, 302 bp, Canis
gatattgtcatgacacagaccccactgtccctgtccgtcagccctggagagccggcctccatctcctgcaaggccagtcagagcctcctgcacagtaatgggaacacctatttgtattggttccgacagaagccaggccagtctccacagcgtttgatctataaggtctccaacagagaccctggggtcccagacaggttcagtggcagcgggtcagggacagatttcaccctgagaatcagcagagtggaggctgatgatgctggagtttattactgcgggcaaggtatacaagatcctcc

SEQ ID NO: 29 - DNA, 302 bp, Canis
gatattgtcatgacacagacgccaccgtccctgtctgtcagccctagagagacggcctccatctcctgcaaggccagtcagagcctcctgcacagtgatggaaacacctatttggattggtacctgcaaaagccaggccagtctccacagcttctgatctacttggtttccaaccgcttcactggcgtgtcagacaggttcagtggcagcgggtcagggacagatttcaccctgagaatcagcagagtggaggctaacgatactggagtttattactgcgggcaaggtacacagcttcctcc

SEQ ID NO: 30 - DNA, 178 bp, Canis
gatattgtcatgacacagaacccactgtccctgtccgtcagccctggagagacggcctccatctcctgcaaggccagtcagagcctcctgcacagtaacgggaacacctatttgaattggttccgacagaagccaggccagtctccacagggcctgatctataaggtctccaacagag

SEQ ID NO: 31 - DNA, 20 bp, Canis
ctgggggccagtgggaaaac

SEQ ID NO: 32 - DNA, 20 bp, Canis
gcacaccgctggtcaaggag

SEQ ID NO: 33 - DNA, 20 bp, Canis
gcactgtcaccatgctgctg

SEQ ID NO: 34 - DNA, 20 bp, Canis
cagatgtgctgtccttgccg

SEQ ID NO: 35 - DNA, 20 bp, Canis
gggtcaactggctgctcatg

SEQ ID NO: 36 - DNA, 20 bp, Canis
cagaggctcagcgggaacac

SEQ ID NO: 37 - DNA, 20 bp, Canis
ggtagctgctggccgtgtac

SEQ ID NO: 38 - DNA, 20 bp, Canis
gccctggatgatggtgctgc

SEQ ID NO: 39 - DNA, 20 bp, Canis
tcagaggagggcgggaagag

SEQ ID NO: 40 - DNA, 20 bp, Canis
ctgcttggagggcttggtgg

SEQ ID NO: 41 - DNA, 21 bp, Canis
tcctggatgcctgtgtcttgg

SEQ ID NO: 42 - DNA, 20 bp, Canis
ggtgctgctgaggctgtagg

SEQ ID NO: 43 - DNA, 20 bp, Canis
aaatagacggctggctgggc

SEQ ID NO: 44 - DNA, 21 bp, Canis
gaacacctgagaggtcgccac

SEQ ID NO: 45 - DNA, 23 bp, Canis
cctggttgttgattgcactgagg

SEQ ID NO: 46 - DNA, 22 bp, Canis
tgatgagggggaagaggtttgg

SEQ ID NO: 47 - DNA, 20 bp, Canis
gtttccagggggtggacagg

SEQ ID NO: 48 - DNA, 21 bp, Canis
ccatctgggcctgtgactctg

SEQ ID NO: 49 - DNA, 23 bp, Canis
ccttacatcctgagaccaagggg